(19) United States
(12) Patent Application Publication
(10) Pub. No.: US 2024/0426843 A1
(43) Pub. Date: Dec. 26, 2024

(54) COMPOSITIONS AND METHODS FOR DIAGNOSING AND TREATING PARKINSON'S DISEASE

(71) Applicants: Monell Chemical Senses Center, Philadelphia, PA (US); The Trustees of the University of Pennsylvania, Philadelphia, PA (US); The United States Government as represented by the Department of Veterans Affairs

(72) Inventors: Pamela Dalton, Philadelphia, PA (US); Hong Wang, Havertown, PA (US); James Morely, Bryn Mawr, PA (US); Noam Cohen, Bala Cynwyd, PA (US)

(21) Appl. No.: 18/705,186

(22) PCT Filed: Oct. 26, 2022
(86) PCT No.: PCT/US2022/078730

§ 371 Date: Apr. 26, 2024

Related U.S. Application Data
(60) Provisional application No. 63/271,989, filed on Oct. 26, 2021.

(51) Int. Cl. G01N 33/68

(52) U.S. Cl. CPC G01N 33/6896; G01N 33/6863; G01N 2333/4728; G01N 2800/2835

(57) ABSTRACT

Provided herein are compositions and methods for diagnosing and treating Parkinson's Disease by the use of one or more of protein biomarkers, e.g., protein expression profile or ratio thereof, from olfactory mucus sample of a subject which is characteristic of PD.

### BACKGROUND OF THE INVENTION

Parkinson's Disease (PD) is a debilitating neurodegenerative disease that progressively robs the patient of independence. As a movement disorder, PD is characterized by muscular rigidity, tremor while resting, loss of balance and aberrant gait. Non-motor symptoms include cognitive decline, sleep disturbance, gastric and bowel dysfunction, such as constipation, as well as sensory disturbances, including loss of vision and smell (Wooten, G. F., Neurochemistry and neuropharmacology of Parkinson's disease, in Movement disorders: neurologic principles and practice, R. L. Watts and W. Koller, Editors. 1997, McGraw-Hill: New York. p. 153-160).

PD is the second most common neurodegenerative disease, and the fastest growing cause of neurological disability. About 10 million people worldwide are currently living with PD, including approximately one million Americans (Yang, W., et al., Current and projected future economic burden of Parkinson's disease in the U.S. NPJ Parkinsons Dis, 2020. 6:15; Marras, C., et al., Prevalence of Parkinson's disease across North America. NPJ Parkinsons Dis, 2018. 4:21). Each year, an additional 60,000 are diagnosed with PD in the U.S. The incidence and prevalence of PD increase with age, with a nearly exponential increase in incidence between ages 55 and 79 (Table 1) (Van Den Eeden, S. K., et al., Incidence of Parkinson's disease: variation by age, gender, and race/ethnicity. Am J Epidemiol, 2003. 157(11):1015-22; Lewin Group, Economic Burden and Future Impact of Parkinson's Disease michaeljfox.org/sites/default/files/media/document/2019% 20Parkinson%27s%20Economic%20Burden%20Study%20-%20FINAL.pdf, 2019; Driver, J. A., et al., Incidence and remaining lifetime risk of Parkinson disease in advanced age. Neurology, 2009. 72(5):432-8). As the US population ages, the prevalence of PD is expected to double by 2030 (Martinez-Martin, P., et al., The long-term direct and indirect economic burden among Parkinson's disease caregivers in the United States. Mov Disord, 2019. 34(2):236-245). The total cost of PD to patients, families, and the US government is estimated at $51.9 billion annually, including direct medical costs of $25.4 billion and indirect/non-medical costs of $26.5 billion (Yang, W., et al., 2020).

Currently, there is no cure for PD. Treatments for PD only offer temporary relief of symptoms. Dopamine replacement therapy (e.g., levodopa) is the standard treatment for symptomatic relief of motor deficits (Ferrazzoli, D., et al., Dopamine Replacement Therapy, Learning and Reward Prediction in Parkinson's Disease: Implications for Rehabilitation. Front Behav Neurosci, 2016. 10:121). However, long-term use of dopamine replacement drugs causes undesirable adverse side effects and/or loss of efficacy. PD is progressive, with worsening of motor and nonmotor symptoms overtime (Wooten et al., 1997). Disease modifying therapies (i.e., therapies that slow or stop disease progression) offer hopes for PD patients. Although many disease modifying therapies are currently in various stages of clinical trials (Elkouzi, A., et al., Emerging therapies in Parkinson disease—repurposed drugs and new approaches. Nat Rev Neurol, 2019. 15(4):204-223; Fields, C.R., N. Bengoa-Vergniory, and R. Wade-Martins, Targeting Alpha-Synuclein as a Therapy for Parkinson's Disease. Front Mol Neurosci, 2019. 12:299; Kalia, L. V., S. K. Kalia, and A. E. Lang, Disease-modifying strategies for Parkinson's disease. Mov Disord, 2015. 30(11):1442-50), they are not yet available to patients. In fact, multiple recent clinical trials for disease modifying therapies have failed, leading many to believe that successful disease modification requires identification of patients at the earliest stages of disease when it is still amenable to neuroprotective therapies (Tropea, T. F. and A. S. Chen-Plotkin, Unlocking the mystery of biomarkers: A brief introduction, challenges and opportunities in Parkinson Disease. Parkinsonism Relat Disord, 2018. 46 Suppl 1: S15-S18; Stephenson, D., et al., The Qualification of an Enrichment Biomarker for Clinical Trials Targeting Early-stages of Parkinson's Disease. J Parkinsons Dis, 2019. 9(3):553-563).

There is an unmet need for early diagnosis of PD. Early diagnosis of PD, before the development of advanced symptoms, may provide a better opportunity to slow or stop disease progression together with disease modifying therapies or other interventions (such as exercise (Crotty, G. F. and M. A. Schwarzschild, Chasing Protection in Parkinson's Disease: Does Exercise Reduce Risk and Progression? Front Aging Neurosci, 2020. 12:186)). Therefore, early identification of PD patients is critically important. The definitive diagnosis of PD requires autopsy. Thus, diagnosis requires clinical evaluation of motor skills. When in doubt, as during the earlier prodromal stages, dopamine transporter imaging (DaTscan) is an adjunct to assist in diagnosis by clinical evaluation. However, it is expensive and time-consuming with a safety issue from the use of a radioactive tracer dye. Furthermore, the imaging lacks specificity; and is qualitative, not suitable for monitoring disease progression. The alternative is real-time quaking-induced conversion (RT-QuIC), which requires a cerebrospinal fluid sample. In addition to being invasive (a safety issue), the assay also lacks specificity. Neither assays are broadly available nor suited for patient screening. Consequently, a simple, affordable, and safer assay for early detection and treatment to prevent disease progression that minimizes symptoms and suffering is an urgent, unmet need.

Olfactory loss is one of the earliest symptoms of PD, often developing before the onset of motor symptoms. Odors are detected, in the first instance, by olfactory sensory neurons (OSNs) within the olfactory epithelium of the nose. These neurons synapse with mitral/tufted cells in the olfactory bulb which, in turn, project to higher order structures in the brain. OSNs are peripherally located, exposed to the external environment and regenerate after injury, features that make them attractive for biomedical sampling. Hypotheses that environmental toxin exposures through the nose contribute to the development of PD suggest that biomarkers unique to PD may be detected in the mucus collected from the olfactory cleft.

There still remains an urgent need in the art for compositions (i.e., safe and affordable tests) useful for diagnosing the existence, evaluating progression and further treating Parkinson's Disease, more specifically, early diagnosis of the disease.

### SUMMARY OF THE INVENTION

In one aspect, provided herein is a composition for diagnosing existence or evaluating progression of Parkinson's Disease in a subject in need thereof, wherein the composition comprises at least one ligand, wherein the at least one ligand is specific for and binds to a protein biomarker in a sample collected from a subject, wherein the protein biomarker is selected from BDNF, EGF, EOTAXIN, FGF2, HGF, IFNa, IFNg, IL1RA, IL10, IL12P40, IL13, IL6, IL8/CXCL8, IP10, MCP-1/CCL2, MIG, MIP1A, MIP1B, RANTES, VEGFa, alpha-Synuclein (α-Syn), Park7/DJ-1 (DJ-1), Park5/UCHL1, LRRK2, Mapt (i.e., tau), Parkin, PINK1, GBA, S100A8, S100A9, Histone H4, Transthyretin, hemoglobin subunit delta, hemoglobin subunit alpha, hemoglobin subunit beta, Glutathion S-transferase P, Cystatin-A, PEBP1, HSPA2, TUBB3, DNAL1, and Krt14, and wherein the ligand is for measuring the expression level of the protein biomarker. In certain embodiments, the composition comprises multiple ligands, wherein each ligand is specific for and binding to a different protein biomarker selected from BDNF, EGF, EOTAXIN, FGF2, HGF, IFNa, IFNg, IL1RA, IL10, IL12P40, IL13, IL6, IL8/CXCL8, IP10, MCP-1/CCL2, MIG, MIP1A, MIP1B, RANTES, VEGFa, alpha-Synuclein (α-Syn), Park7/DJ-1 (DJ-1), Park5/UCHL1, LRRK2, Mapt (i.e., tau), Parkin, PINK1, GBA, S100A8, S100A9, Histone H4, Transthyretin, hemoglobin subunit delta, hemoglobin subunit alpha, hemoglobin subunit beta, Glutathione S-transferase P, Cystatin-A, PEBP1, HSPA2, TUBB3, DNAL1, and Krt14. In certain embodiments, the collected sample is an olfactory cleft sample. In certain embodiments, the at least one ligand is attached to a detectable label, optionally wherein each of the ligands is attached to a different detectable label. In certain embodiments, the protein biomarker is α-Syn. In certain embodiments, the protein biomarker is DJ-1. In certain embodiments, the protein biomarker is MCP-1. In some embodiments, the composition comprises ligands which are specific for and bind protein biomarkers of α-Syn and DJ-1 for use in calculating the α-Syn/DJ-1 ratio.

In another aspect, provided herein is a kit comprising composition for diagnosing existence or evaluating progression of PD in a subject in need thereof. In certain embodiments, the kit further comprises an apparatus for sample collection. In certain embodiments, the kit comprises an apparatus for sample collection which is a polypropylene sponge for mucus collection, a tube for storage of the sponge, optionally wherein the tube contains a reagent which stabilizes the mucus sample. In certain embodiments, the kit comprises an apparatus for sample collection which is s nasal swab with soft, flexible, and absorbing tips.

In a further aspect, provided herein is a method of diagnosing existence or evaluating progression of Parkinson's Disease (PD) in a subject, wherein the method comprising: (i) measuring an expression level of one or more protein biomarker in the subject, wherein the one or more protein biomarker is selected from BDNF, EGF, EOTAXIN, FGF2, HGF, IFNa, IFNg, IL1RA, IL10, IL12P40, IL13, IL6, IL8/CXCL8, IP10, MCP-1/CCL2, MIG, MIP1A, MIP1B, RANTES, VEGFa, alpha-Synuclein (α-Syn), Park7/DJ-1 (DJ-1), Park5/UCHL1, LRRK2, Mapt (tau), Parkin, PINK1, GBA, S100A8, S100A9, Histone H4, Transthyretin, hemoglobin subunit delta, hemoglobin subunit alpha, hemoglobin subunit beta, Glutathione S-transferase P, Cystatin-A, PEBP1, HSPA2, TUBB3, DNAL1, and Krt14; (ii) comparing the expression level of the protein biomarker in the subject to a reference or control expression level for the at least one or more or each biomarker; and (iii) diagnosing PD in the subject on the basis of the comparison, wherein the change in the expression level of protein biomarker or the ratio thereof of the subject's expression level from those of reference or control expression level correlates with a diagnostic evaluation of PD. In certain embodiments, the method comprises measuring the expression level of the one or more protein biomarker in a sample obtained from the subject, wherein the sample is an olfactory cleft mucus sample. In certain embodiments, the method comprises measuring and comparing the expression level of the one or more protein biomarker which is alpha-synuclein (α-Syn), DJ-1, and/or MCP1. In certain embodiments, the method comprises measuring expression level and comparing the expression level ratio of α-Syn/DJ-1. In certain embodiments, the ratio of α-Syn/DJ-1 of greater than 1.5 correlates with a diagnosis of PD in the subject. In certain embodiments, the method comprises measuring expression level of the one or more protein biomarker using a Western Blot (WB), a Luminex-based multiplex immunoassay, an enzyme linked immunoabsorbent assay (ELISA), an immunoprecipitation assay, a complement fixation assay, a fluorescence activated cell sorter (FACS), a protein chip, or a Liquid Chromatography with tandem mass spectrometry (LC-MS/MS). In certain embodiments, the method comprises diagnosing PD in subject at an early-stage of PD.

In yet another aspect, provided herein is a method of diagnosing and treating Parkinson's Disease (PD) in a subject, the method comprising: (i) obtaining an olfactory mucus sample from a subject; (ii) measuring an expression level of one or more protein biomarker in the subject, wherein the one or more protein biomarker is selected from BDNF, EGF, EOTAXIN, FGF2, HGF, IFNa, IFNg, IL1RA, IL10, IL12P40, IL13, IL6, IL8/CXCL8, IP10, MCP-1/CCL2, MIG, MIP1A, MIP1B, RANTES, VEGFa, alpha-Synuclein (α-Syn), Park7/DJ-1 (DJ-1), Park5/UCHL1, LRRK2, Mapt (tau), Parkin, PINK1, GBA, S100A8, S100A9, Histone H4, Transthyretin, hemoglobin subunit delta, hemoglobin subunit alpha, hemoglobin subunit beta, Glutathione S-transferase P, Cystatin-A, PEBP1, HSPA2, TUBB3, DNAL1, and Krt14; (iii) comparing the expression level of the protein biomarker in the subject to a reference or control expression level for the at least one or more or each biomarker; (iv) diagnosing PD in the subject on the basis of the comparison, wherein the change in the expression level of protein biomarker or the ratio thereof of the subject's expression level from those of reference or control expression level correlates with a diagnostic evaluation of PD, optionally wherein the PD diagnosis is an early-stage PD; and (v) treating the subject with PD modifying therapies. In certain embodiments, the method comprises is measuring and comparing the expression level of alpha-synuclein (α-Syn), DJ-1, and/or MCP1 in the subject to a reference or control expression level for the at least one or more or each biomarker. In certain embodiments, the method comprises comparing the expression level ratio of α-Syn/DJ-1 in the subject to a reference or control expression level ratio for the at least one or more or each biomarker. In certain embodiments, the method comprises introducing PD modifying therapies which prevent and/or alleviate PD related symptoms including essential tremor, multiple system atrophy and progressive supranuclear palsy.

Other aspects and advantages of the invention will be readily apparent from the following detailed description of the invention.

### BRIEF DESCRIPTION OF THE DRAWINGS

FIG. 1A shows the levels of α-SYN in the olfactory mucus from PD patients and control subjects, with P values from t tests are shown.

FIG. 1B shows the levels of DJ-1 in the olfactory mucus from PD patients and control subjects, with P values from t tests are shown.

FIG. 1C shows the ratio of levels α-SYN/DJ-1 in the olfactory mucus from PD patients and control subjects, with P values from t tests are shown.

FIG. 2 shows the levels of MCP-1 in the olfactory mucus from PD patients and control subjects. P value from t test is shown.

FIG. 3 shows ROC curve of α-Syn/DJ-1 ratio in olfactory mucus; this ratio shows high biomarker potential for PD. AUC=0.907. p=0.0018.

FIG. 4A shows Levels of DJ-1 in olfactory mucus decline in elderly subjects. Olfactory mucus proteins were analyzed by LC-MS/MS. N=12 for each group. Young: 21-40 years old. Elderly: 65-80 years old. **p=0.004.

FIG. 4B shows levels of α-Syn are increased in the olfactory mucus of PD patients; N=12 for PD patients; N=9 for age-matched controls; and p=0.096 (Mann-Whitney test, two-tailed).

FIG. 4C shows the α-Syn/DJ-1 ratio is markedly elevated in olfactory mucus of PD patients; N=12 for PD patients; N=9 for age-matched controls; and ***p=0.0009 (Mann-Whitney test, two-tailed).

FIG. 5A shows inverse correlation between IL-8 levels and combined olfactory threshold scores for n-butanol and phenylethyl alcohol; correlation coefficients (r) and p values are indicated in each panel.

FIG. 5B shows inverse correlation between MCP-1 levels and combined olfactory threshold scores for n-butanol and phenylethyl alcohol; correlation coefficients (r) and p values are indicated in each panel.

FIG. 6 shows that levels of MCP-1 are significantly increased in the olfactory mucus of PD patients; N=12 for PD patients; N=9 for age-matched controls; and *p=0.023 (Mann-Whitney test, two-tailed).

FIG. 7 shows schematic representation of the timeline of the project.

FIG. 8 shows schematic representation of sample collection, data acquisition, and algorithm optimization.

### DETAILED DESCRIPTION OF THE INVENTION

Provided herein are composition and methods of diagnosing existence and evaluating progression Parkinson's Disease (PD) in a subject. Also provided herein are compositions and methods of diagnosing existence and treating PD in a subject at an early-stage of PD. Additionally provided herein is a kit comprising the composition for diagnosing existence and evaluating progression of PD, and further comprising apparatus for sample collection. The compositions and methods described herein provide the ability to distinguish the olfactory cleft sample collected from a subject with PD and without PD, by determining a characteristic protein expression profile of the protein biomarker in a subject. The characteristic protein expression profile is compared with the profile of one or more subjects of the same class, e.g., subjects having or not having PD, and a control group. to see which class the protein biomarker expression profile is most similar, to provide a useful diagnosis of PD, more specifically at an early-stage PD. The diagnosis allows for early intervention and treatment of PD.

Current standard of care for diagnosing PD comprises typically a diagnosis by neurologists through clinical evaluations based on symptoms, medical history, and neurological exams. Clinical criteria for the diagnosis of PD, such as the UK Brain Bank Criteria or the Movement Disorder Society PD Criteria, demand motor deficits, such as bradykinesia plus rigidity, resting tremor, or postural instability (Reichmann, H., Clinical criteria for the diagnosis of Parkinson's disease. Neurodegener Dis, 2010. 7(5): p. 284-90; Postuma, R. B., et al., MDS clinical diagnostic criteria for Parkinson's disease. Mov Disord, 2015. 30(12): p. 1591-601). Compared to the gold standard pathological diagnosis (i.e., presence of Lewy pathology in histological examinations at death), misdiagnosis of PD ranges from 10 to 50% (Hughes, A. J., S. E. Daniel, and A. J. Lees, Improved accuracy of clinical diagnosis of Lewy body Parkinson's disease. Neurology, 2001. 57(8): p. 1497-9; Marshall, V. L., et al., Parkinson's disease is over-diagnosed clinically at baseline in diagnostically uncertain cases: a 3-year European multicenter study with repeat [123I]FP-CIT SPECT. Mov Disord, 2009. 24(4): p. 500-8). Current methods of diagnosis comprise those requiring physical manifestation of symptoms, which does not allow for early-stage PD diagnosis that is useful for increased PD therapy efficacy. Furthermore, for difficult to diagnose cases, doctors may order DaTscan to differentiate neurodegenerative Parkinsonian syndrome from non-dopamine deficiency etiologies of Parkinsonism. DaTscan is an FDA-approved brain imaging test used as an adjunct to other diagnostic evaluations of adult patients with suspected Parkinsonian syndrome (PS). DaTscan is a radiopharmaceutical indicated for striatal dopamine transporter visualization using single photon emission computed tomography (SPECT) brain imaging. It has the advantage of directly visualizing the brain area involved in PD and may be able to detect prodromal PD (Jennings, D., et al., Conversion to Parkinson Disease in the PARS Hyposmic and Dopamine Transporter-Deficit Prodromal Cohort. JAMA Neurol, 2017. 74(8): p. 933-940). However, it is unlikely to become a broadly used screening tool for identifying early PD, as it is costly (ranging from $2500-$5000 per test) and time consuming (requiring several hours from time of injection of the radiotracer to imaging). The test also has safety issues associated with a radioactive tracer dye. Additionally, RT-QuIC test may be used to diagnose PD. RT-QuIC was developed as a method to detect misfolded proteins that can self-amplify, such as the prion protein (Dong, T. T. and K. Satoh, The Latest Research on RT-QuIC Assays-A Literature Review. Pathogens, 2021. 10(3)). Misfolded α-Syn, implicated in PD, can also propagate in vivo and in vitro and therefore can be detected by RT-QuIC method. Multiple studies have reported that RT-QuIC can identify PD patients with high sensitivity (ranging from 65%-97%) and specificity (ranging from 84%-100%) (Dong, T. T. and K. Satoh, The Latest Research on RT-QuIC Assays-A Literature Review. Pathogens, 2021. 10(3); Fairfoul, G., et al., Alpha-synuclein RT-QuIC in the CSF of patients with alpha-synucleinopathies. Ann Clin Transl Neurol, 2016. 3(10): p. 812-818; Saijo, E., et al., Ultrasensitive RT-QuIC Seed Amplification Assays for Disease-Associated Tau, alpha-Synuclein, and Prion Aggregates. Methods Mol Biol, 2019. 1873: p. 19-37). In 2019, FDA granted Amprion with breakthrough device designation for the detection of α-Syn by RT-QuIC method. Amprion launched SYNTap Biomarker Test™ as a Lab Developed Test in October 2021. However, the test lacks diagnostic specificity, as misfolded α-Syn is implicated not only in PD but also other neurological disorders, such as multiple system atrophy and dementia with Lewy bodies. Additionally, RT-QuIC is qualitative instead of quantitative; and thus not be useful for monitoring disease progression. Furthermore, invasive spinal tap is required for CSF sampling, a potential safety issue that will limit development as a screening test for PD.

In comparison to the above-mentioned standard of care, the compositions, kits, and methods for diagnosing and evaluating progression of PD as described herein provide an advantage in early detection based on protein biomarker directly connected to PD pathogenesis. More specifically, the compositions, kit and methods described herein also provide for the opportunity to identify PD patients early for intervention in the disease process, that could lead to delay or prevention of PD symptoms. Additionally, the compositions, kit and methods described herein provide for a safer, minimally invasive collection of olfactory mucus using polypropene sponge or nasal swab, and a more cost-effective and affordable alternative. The compositions, kit and methods described herein provide for simpler assay requiring less skilled personnel, and is/are more compatible with commercial clinical lab procedures. The compositions, kit and methods described herein also provide for reliable assay that is sensitive and specific, involving well-established and reproducible Luminex-based immunoassay, commonly used by clinical labs to detect other biomarkers.

As used herein, the term “patient” or “subject” as used herein means a mammalian animal, including a human, a veterinary or farm animal, a domestic animal or pet normally used for clinical research. In one embodiment, the subject of these methods and compositions is a human.

As used herein, terms “disease”, “disorder”, and “condition” are used interchangeably, as to indicate an abnormal state in subject. A used herein, the disease is Parkinson's Disease (PD). PD is a movement disorder, and is characterized by muscular rigidity, tremor while resting, loss of balance and aberrant gait. Additionally, PD may be characterized by non-motor symptoms, which include cognitive decline, sleep disturbance, gastric and bowel dysfunction, such as constipation, as well as sensory disturbances, including loss of vision and smell (Wooten, G. F., Neurochemistry and neuropharmacology of Parkinson's disease, in Movement disorders: neurologic principles and practice, R. L. Watts and W. Koller, Editors. 1997, McGraw-Hill: New York, 153-160).

As used herein “control” or “control subject” refers to the source of the reference protein expression profiles as well as the particular panel of control subjects described herein. In one embodiment, the control or reference level is from a single subject. In another embodiment, the control or reference level is from a population of individuals sharing a specific characteristic. In yet another embodiment, the control or reference level is an assigned value which correlates with the level of a specific control individual or population, although not necessarily measured at the time of assaying the test subject's sample. In one embodiment, the control subject or reference is from a patient (or population) having a PD. In one embodiment, the control subject or reference is from a patient (or population) not having a PD (i.e., healthy control). In one embodiment, the control subject or reference is from a patient (or population) having an early-stage PD.

As used herein, the term “sample” refers to any biological fluid or tissue that contains olfactory cleft mucus (olfactory mucus). The most suitable sample for use in this invention includes mucus collected from nasal cavity. The term “olfactory mucus” refers to a biofluid covering the surface of the olfactory sensory neurons. In certain embodiments, the sample is collected by placing a polypropylene sponge in nasal the cavity. In certain embodiments, the sample is collected by nasal brushings. In certain embodiments, the sample may be collected using a nasal swab. Such samples may further be diluted with saline, buffer or a physiologically acceptable diluent, or admixed with stabilizing solution. Alternatively, such samples can be concentrated by conventional means.

By “diagnosis” or “evaluation” it is meant a diagnosis of PD, a diagnosis of a stage of PD, a diagnosis and evaluation of progression of PD, or an evaluation of the response of a PD-modifying therapy. As used throughout application, the term “diagnosis” refers to identifying the presence or characteristic of a pathological condition which is Parkinson's disease. The current description includes predicting the onset and progression of Parkinson's disease. More specifically, an early-stage diagnosis and/or evaluation of Parkinson's disease is described.

As used herein, “sensitivity” (also called the true positive rate), measures the proportion of positives that are correctly identified as such (e.g., the percentage of sick people who are correctly identified as having the condition).

As used herein “biomarker” refers to protein biomarker the expression level of which changes (either upregulated or downregulated) characteristically in the presence of PD. A statistically significant number if such protein biomarkers thus form suitable biomarker expression profile for use in the methods and compositions. Such biomarkers include BDNF, EGF, EOTAXIN, FGF2, HGF, IFNa, IFNg, IL1RA, IL10, IL12P40, IL13, IL6, IL8/CXCL8, IP10, MCP-1/CCL2, MIG, MIP1A, MIP1B, RANTES, VEGFa, α-Synuclein (α-SYN), Park7/DJ-1, Park5/UCHL1, LRRK2, Mapt (i.e., tau), Parkin, PINK1, GBA, S100A8, S100A9, Histone H4, Transthyretin, hemoglobin subunit delta, hemoglobin subunit alpha, hemoglobin subunit beta, Glutathione S-transferase P, Cystatin-A, PEBP1, HSPA2, TUBB3, DNAL1, and Krt14. In certain embodiments, the ratio of biomarker expression is used. In certain embodiments, the ratio of α-SYN/DJ-1 is used.

As used herein, “labels” or “reporter molecules” are chemical or biochemical moieties useful for labeling a nucleic acid (including a single nucleotide), polynucleotide, oligonucleotide, or protein ligand, e.g., amino acid or antibody. “Labels” and “reporter molecules” include fluorescent agents, chemiluminescent agents, chromogenic agents, quenching agents, radionucleotides, enzymes, substrates, cofactors, inhibitors, magnetic particles, and other moieties known in the art. “Labels” or “reporter molecules” are capable of generating a measurable signal and may be covalently or noncovalently joined or bound to ligand.

### Compositions

In current application, the inventors have shown that protein expression profiles of the olfactory mucus of the PD-diagnosed patients differ significantly from those seen in patients not having PD. The protein expression (i.e., protein biomarker) profile provide for new diagnostic markers for detection of PD, for evaluation of progression of PD, and for detection of early-stage PD. The protein expression profile is evaluated in olfactory mucus samples, which could prevent patients from undergoing expensive and invasive procedures for collection of cerebrospinal fluid (CSF). Since the risks are very low, the benefit to risk ratio is very high. In one embodiment, the methods and compositions described herein may be used in conjunction with clinical risk factors to help physicians make more accurate decisions about how to manage patients at risk for PD. Another advantage of this invention is that diagnosis may occur early since diagnosis is not dependent upon detecting physical manifestations of the PD (i.e., tremors).

Provided herein are pharmaceutical compositions comprising at least one ligand, which is specific for and binds to a protein biomarker in a sample collected from a subject wherein the ligand is for measuring the expression level of the protein biomarker in a sample. In certain embodiments the protein biomarker is selected from BDNF, EGF, EOTAXIN, FGF2, HGF, IFNa, IFNg, IL1RA, IL10, IL 12P40, IL13, IL6, IL8/CXCL8, IP10, MCP-1/CCL2, MIG, MIP1A, MIP1B, RANTES, VEGFa, alpha-Synuclein (α-Syn), Park7/DJ-1 (DJ-1), Park5/UCHL1, LRRK2, Mapt (i.e., tau), Parkin, PINK1, GBA, S100A8, S100A9, Histone H4, Transthyretin, hemoglobin subunit delta, hemoglobin subunit alpha, hemoglobin subunit beta, Glutathione S-transferase P, Cystatin-A, PEBP1, HSPA2, TUBB3, DNAL1, and Krt14. In certain embodiments, the composition comprises multiple ligands, each of which is specific for the protein biomarkers described herein.

In certain embodiments, the sample is an olfactory mucus sample (i.e., olfactory cleft mucus sample). In certain embodiments, the olfactory mucus sample is collected by placing a sponge in a nasal cavity. In certain embodiment, the olfactory mucus sample is collected by a nasal swab. In some embodiments, other methods of olfactory mucus collection may be used in which the collected olfactory mucus sample is diluted (i.e., nasal lavage). In certain embodiments, other methods of olfactory mucus collection may be used in which the collected olfactory mucus sample is not diluted (i.e., not a nasal lavage). In certain embodiments, the olfactory mucus sample is a fresh sample. In certain embodiments, the olfactory mucus sample is a previously collected and stored sample (e.g., olfactory mucus admixed with stabilizing solution, optionally stored at room temperature, 4° C., −20° C., or −80° C.).

In certain embodiments, the composition comprises at least one, at least two, at least three, at least four, at least five ligand/s which is/are specific for and binds to protein biomarker/s in a sample. In one embodiment, the composition comprises a ligand which is specific for and binds to a protein biomarker which is α-Syn. In one embodiment, the composition comprises ligand which is specific for and binds to a protein biomarker which is DJ-1. In one embodiment, the composition comprises ligand which is specific for and binds to a protein biomarker which is MCP-1. In one embodiment, the composition comprises ligands which are specific for and binds to protein biomarkers which are α-Syn and DJ-1. In certain embodiments, the composition comprises ligands which are specific for and binds to protein biomarker which are α-Syn, DJ-1, and MCP-1.

In certain embodiments, the ligand is attached to a detectable label. In certain embodiments, more than one ligand is used, where each of the ligands is attached to a different detectable label.

In one embodiment, a novel protein expression profile can identify and distinguish patients having PD and not having PD. In some embodiments, a novel protein expression profile can identify and distinguish patients having early-stage PD.

### Methods and Kits

In one aspect, provided herein is a method of diagnosing existence or evaluating progression of Parkinson's Disease (PD) in a subject using a composition as described herein. In another aspect, provided herein is a method of diagnosing and treating PD in a subject in need thereof.

In certain embodiments, the method comprises obtaining an olfactory mucus sample from a subject; measuring an expression level of one or more protein biomarker in the subject, wherein the one or more protein biomarker is selected from BDNF, EGF, EOTAXIN, FGF2, HGF, IFNa, IFNg, IL1RA, IL10, IL12P40, IL13, IL6, IL8/CXCL8, IP10, MCP-1/CCL2, MIG, MIP1A, MIP1B, RANTES, VEGFa, alpha-Synuclein (α-Syn), Park7/DJ-1 (DJ-1), Park5/UCHL1, LRRK2, Mapt (tau), Parkin, PINK1, GBA, S100A8, S100A9, Histone H4, Transthyretin, hemoglobin subunit delta, hemoglobin subunit alpha, hemoglobin subunit beta, Glutathione S-transferase P, Cystatin-A, PEBP1, HSPA2, TUBB3, DNAL1, and Krt14; comparing the expression level of the protein biomarker in the subject to a reference or control expression level for the at least one or more or each biomarker; and diagnosing PD in the subject on the basis of the comparison, wherein the change in the expression level of protein biomarker or the ratio thereof of the subject's expression level from those of reference or control expression level correlates with a diagnostic evaluation of PD.

In certain embodiments the method comprises obtaining an olfactory mucus sample from a subject; measuring an expression level of one or more protein biomarker in the subject, wherein the one or more protein biomarker is selected from BDNF, EGF, EOTAXIN, FGF2, HGF, IFNa, IFNg, IL1RA, IL10, IL12P40, IL13, IL6, IL8/CXCL8, IP10, MCP-1/CCL2, MIG, MIP1A, MIP1B, RANTES, VEGFa, alpha-Synuclein (α-Syn), Park7/DJ-1 (DJ-1), Park5/UCHL1, LRRK2, Mapt (tau), Parkin, PINK1, GBA, S100A8, S100A9, Histone H4, Transthyretin, hemoglobin subunit delta, hemoglobin subunit alpha, hemoglobin subunit beta, Glutathione S-transferase P, Cystatin-A, PEBP1, HSPA2, TUBB3, DNAL1, and Krt14; comparing the expression level of the protein biomarker in the subject to a reference or control expression level for the at least one or more or each biomarker; diagnosing PD in the subject on the basis of the comparison, wherein the change in the expression level of protein biomarker or the ratio thereof of the subject's expression level from those of reference or control expression level correlates with a diagnostic evaluation of PD, optionally wherein the PD diagnosis is an early-stage PD; and treating the subject with PD modifying therapies.

In certain embodiment, the method comprises measuring expression levels of alpha-synuclein (α-Syn), DJ-1, and/or MCP1 in the subject and comparing to a reference or control expression level (expression) for the at least one or more or each biomarker. In certain embodiments, the measures expression levels of protein biomarkers are used in calculating ration between two or more biomarker expression levels. In certain embodiments, the ratio of protein biomarker expression level is ratio of α-Syn/DJ-1 expression level. In certain embodiments, the ratio of α-Syn/DJ-1 expression levels of greater than about 1.5 correlates with a diagnosis of PD in the subject. In certain embodiments, the ratio of α-Syn/DJ-I expression levels of greater than about 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6.0, 6.1, 6.2, 6.3, 6.4, 6.5 or greater correlates with a diagnosis of PD in the subject.

In other embodiments, the ratio of α-Syn/DJ-1 expression levels of greater than about 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6.0, 6.1, 6.2, 6.3, 6.4, 6.5 or greater, in conjunction with an elevated MCP-1 expression level, as compared to a control, correlates with a diagnosis of PD in the subject. In certain embodiments, the increase of the MCP-1 expression level in subject in comparison to the expression profile of healthy control is at least about 0.5-fold, at least about 0.75-fold, at least about 1.0-fold, at least about 1.25-fold, at least about 1.5-fold, at least about 1.75-fold, at least about 2.0-fold, and/or at least about 2.5-fold.

In certain embodiments, the measured expression level in a subject relates to the protein expression profile of a healthy control in that α-Syn and MCP-1 expression levels are increased, and DJ-1 expression levels are decreased, which correlated with PD diagnosis in a subject. In certain embodiments, the change in the expression level of protein biomarker or the ratio thereof comprises an upregulation of the one or more of protein biomarker in comparison to said reference or control or a downregulation of one or more selected genes in comparison to said reference or control. In certain embodiments, the change difference (increase or decrease as applicable) of the measured expression level in subject in comparison to the expression profile of healthy control is at least about 0.5-fold, at least about 0.75-fold, at least about 1.0-fold, at least about 1.25-fold, at least about 1.5-fold, at least about 1.75-fold, at least about 2.0-fold, and/or at least about 2.5-fold.

In certain embodiments, a method comprises measuring the expression level of the one or more protein biomarker using a Western Blot (WB), a Luminex-based multiplex immunoassay, an enzyme linked immunoabsorbent assay (ELISA), an immunoprecipitation assay, a complement fixation assay, a fluorescence activated cell sorter (FACS), a protein chip, or a Liquid Chromatography with tandem mass spectrometry (LC-MS/MS).

In certain aspects, the methods further include treating a patient that has been diagnosed with PD. The most widely used treatment, especially at earlier stages, is the dopamine precursor levodopa (L-DOPA). The drug brings the lacking neurotransmitter to the dopaminergic neurons, thus decreasing motor symptoms. Although less effective at improving motor symptoms, dopamine agonists such as ropirole or rotigotine, pergolide, cabergoline, apomorphine or lisuride and monoamine oxidase-B inhibitors (involved in the catabolic breakdown of dopamine) such as selegiline or rasagiline, and catechol-O-methyltransferase (COMT) inhibitors such as entacapone are also commonly used at early stages of the disease. Additionally, adenosine A₂ₐ receptor antagonist or amantadine is used in treatment. In certain embodiments, the methods further include treating a patient diagnosed with PD using compositions, kits and methods described herein, wherein treatment comprises surgical treatment. In certain embodiments, the surgical treatment includes but not limited to deep brain stimulation, focused ultrasound ablation, cardidopa/levodopa infusion pump. In certain embodiments, the treatment further comprises rehabilitative therapy including stretching, strength training, physical, speech, swallow and occupational therapies. In certain embodiments, the treatment comprises targeting α-Syn, more specifically its accumulation and aggregation (i.e., via reduction of α-Syn production or increasing α-Syn clearance). See, Pirotosek, Z., et al., Update in the management of Parkinson's Disease for General Neurologist, Hindawi Parkinson's Disease, 2020, Article ID 9131474; Kobylecki, C., Update on the Diagnosis and management of Parkinson's disease, Clinical Medicine 2020, 20(4):393-398; Stoker, T. B., et al., Emerging Treatment Approaches for Parkinson's Disease, Frontiers in Neuroscience, 2018, 12:1-10 which are incorporated herein by reference in its entirety. See also, U.S. Pat. No. 9,492,410B2 which is incorporated herein by reference in its entirety.

In certain embodiments, the method of diagnosing and treating PD comprises increasing treatment and/or modifying treatment, based on results of the protein expression profile of measured the at least one or more protein biomarkers using compositions and kits as described herein. In certain embodiments, the medication dose and/or timing is modified based on results of the protein expression profile of measured the at least one or more protein biomarkers using compositions and kits as described herein. In certain embodiments, one or more method of treatment is used, based on the results of the protein expression profile. For example, the method of treatment may include (1) continuous subcutaneous infusion of apomorphine; (2) intra-intestinal infusion of levodopacarbidopa gel (LCIG); and (3) deep-brain stimulation (DBS). In certain embodiments, the methods of treatment may be combined based on progression of PD. In certain embodiments, the methods of treatment may be combined to address motor (e.g., tremors) and non-motor symptoms (e.g., blood pressure, tachycardia, sleep disturbance).

In a further aspect, a kit is provided herein comprising compositions as described herein which further comprises apparatus for sample collection. In certain embodiments, the apparatus for sample collection comprises a polypropylene sponge for mucus collection, a tube for storage of the sponge, optionally wherein the tube contains a reagent which stabilizes the mucus sample. In certain embodiments, the apparatus for sample collection comprises a nasal swab with soft, flexible and absorbing tips, and tube for storage of sample, optionally wherein the nasal swab comprises a flexible tip which may be broken off and placed in a tube for easier storage. In certain embodiments, a nasal swab is placed close to the olfactory groove between the middle turbinate and superior nasal septum in one nostril, after 10 min, the swab is removed from the nose, and the absorbing tip will be cut off or broken off, placed in a microfuge tube, and centrifuged at 10,000 rpm for 2 min to collect the olfactory mucus, mucus then aliquoted, and optionally snap-frozen on dry ice, and stored at −80° C.

As used herein, the term “administration” or any grammatical variations thereof refers to delivery of composition described herein to a subject.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be included and described using “consisting of” or “consisting essentially of” language. As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

It is to be noted that the term “a” or “an” refers to one or more. As such, the terms “a” (or “an”), “one or more,” and “at least one” are used interchangeably herein.

As used herein, the term “about” or “˜” refers to a variant of ±10% from the reference integer and values therebetween, unless otherwise specified. For example, “about” 500 μM includes ±50 (i.e., 450-550, which includes the integers therebetween). For other values, particularly when reference is to a percentage (e.g., 90% of taste), the term “about” is inclusive of all values within the range including both the integer and fractions.

As used herein, the term “about” means a variability of plus or minus 10% from the reference given, unless otherwise specified.

In certain instances, the term “E+#” or the term “e+#” is used to reference an exponent. For example, “5E10” or “5e10” is 5×10¹⁰. These terms may be used interchangeably.

With regard to the description of various embodiments herein, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following examples are provided to illustrate certain aspects of the claimed invention. The invention is not limited to these examples.

These examples describe a novel formula of olfactory mucus proteins as biomarkers for Parkinson's Disease (PD). By using highly sensitive and quantitative biochemical assays we have identified some olfactory mucus proteins that show significant correlation with PD.

Briefly, we collected mucus secretions from the olfactory cleft of 12 PD patients and 9 non-PD control subjects. The otorhinolaryngology (ear-nose-throat or ENT) physician administered a small amount of oxymetazoline (decongestant) and lidocaine (topical anesthetic) to one side of the nares. Following this, a small polypropylene sponge was placed in the olfactory cleft under direct endoscopic visualization. The sponge was left in place for 10 minutes after which it was removed, placed into a small vial and centrifuged to obtain the mucus. The mucus was stored at −80° C., and before analysis, it was cleared by centrifugation at 12,000 rpm for 5-10 min to remove cellular debris. Proteins in the collected mucus were analyzed using antibody-based Luminex multiplex magnetic bead assays following the manufacturers' suggested protocols. The biomarkers of interest include BDNF, EGF, EOTAXIN, FGF2, HGF, IFNa, IFNg, IL1RA, IL10, IL12P40, IL 13, IL6, IL8/CXCL8, IP10, MCP-1/CCL2, MIG, MIP1A, MIP1B, RANTES, VEGFa, α-Synuclein (α-SYN), Park7/DJ-1, Park5/UCHL1, LRRK2, Mapt (i.e., tau), Parkin, PINK1, GBA, S100A8, S100A9, Histone H4, Transthyretin, hemoglobin subunit delta, hemoglobin subunit alpha, hemoglobin subunit beta, Glutathion S-transferase P, Cystatin-A, PEBP1, HSPA2, TUBB3, DNAL1, and Krt14.

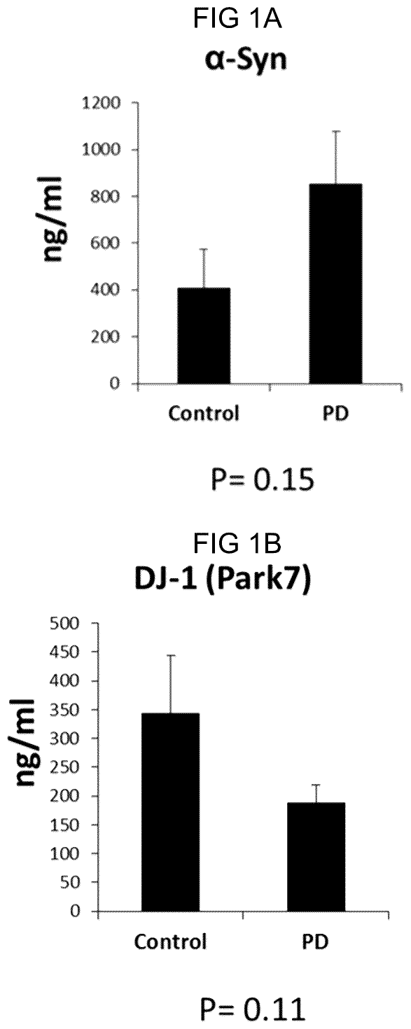

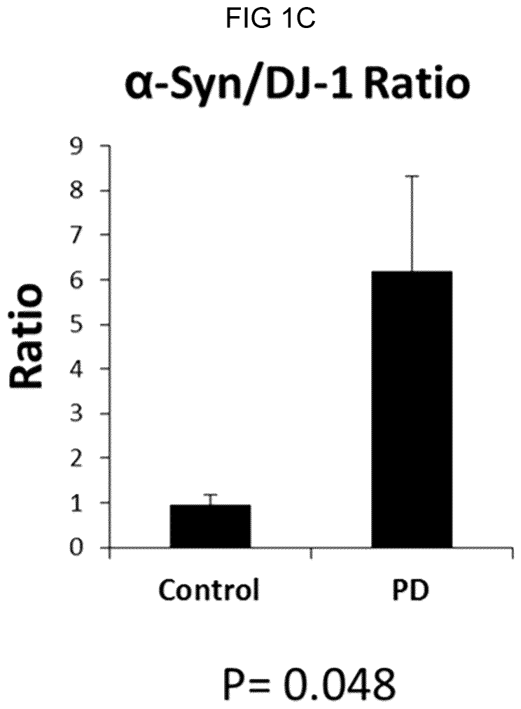

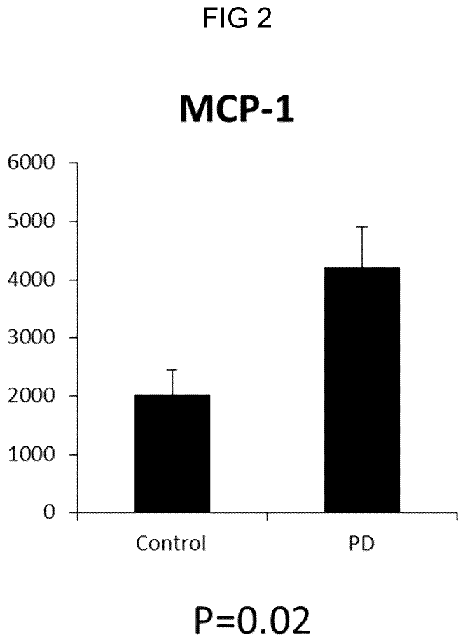

In summary, our results show that there is a trend of increase in the levels of α-SYN in the olfactory mucus from PD patients (FIG. 1A). Similarly, there is a trend of decrease of DJ-1 in the olfactory mucus from PD patients (FIG. 1B). A T-tests comparing α-SYN or DJ-1 levels between PD patients and controls did not show significant differences. However, the ratio of α-SYN to DJ-1 levels was significantly higher in the mucus of PD patients than that of controls (p=0.048, FIG. 1C). The levels of MCP-1, a chemokine related to inflammation, were also significantly higher in the mucus of PD patients than that of controls (p=0.02, FIG. 2). Furthermore, our results show that the olfactory mucus proteins α-SYN, DJ-1, and MCP-1 are potential biomarkers for PD. In particular, the ratio of α-SYN to DJ-1 and the levels of MCP-1 together can be used as a biomarker for the diagnosis of PD.

### Example 1. Technical Background and Context

### (A) Genes and Pathways Involved in PD Pathogenesis.

The first PD causal gene identified through genetic studies of familial PD is the gene encoding α-Syn. Missense mutations in the α-Syn gene result in autosomal dominant form of PD [25, 26]. Multiplications (e.g., duplication or triplication) of the α-Syn gene also lead to familial PD [27, 28], which indicates that higher levels of α-Syn can lead to PD pathogenesis. These genetic data are highly consistent with the role of α-Syn in forming toxic protein aggregates that contribute to neuronal cell death [29, 30]. In addition to α-Syn, genetic studies identified many other PD causal genes, including DJ-1, Parkin, PINK1, and LRRK2. Similar to α-Syn, LRRK2 mutations cause autosomal dominant PD, which suggests that gain-of-function mutations in LRRK2 drive PD pathogenesis [31]. Targeting α-Syn or LRRK2 is a major strategy for developing disease modifying therapies for PD [9-11]. Conversely, mutations in DJ-1, Parkin, and PINK1 are autosomal recessive, which suggests that loss-of-function mutations in these genes lead to PD [32]. Besides these well studied PD causal genes, many other candidates have been identified through recent genome-wide association studies and single-cell multi-omic studies [33, 34]. Genes involved in oxidative stress, mitochondrial dysfunction, abnormal protein processing/degradation, and neuroinflammation all contribute to PD pathogenesis [33, 35, 36]. Because these genes are mechanistically connected to PD, the proteins they encode are potential candidates for PD biomarker development.

Despite of many years of intensive research, diagnostic biomarkers for PD are still limited. As described throughout, there is a special need for diagnosing PD early, preferably before the onsite of motor defects. Early diagnosis and treatment of PD has the potential to slow or alter the course of disease progression. However, to date, safe and affordable diagnostic tests for early/prodromal PD are still lacking.

### (B) The Olfactory System Holds Great Potential for Developing Biomarkers for Early Diagnosis of PD.

The olfactory epithelium is the only accessible peripheral nervous system with highly concentrated sensory neurons. Histopathological studies of PD patients indicate that PD begins in the peripheral nervous systems that frequently encounter toxic or infectious agents, such as in the olfactory and gastrointestinal nervous systems [37, 38]. The pathological hallmarks of PD, α-Syn-containing protein aggregates (i.e., Lewy bodies and neurites, collectively referred to as Lewy pathology), are detected in the olfactory bulb and enteric neurons early in PD pathogenesis [39-44]. Lewy pathology is also found in the olfactory epithelium of PD patients [45, 46], including inside olfactory sensory neurons [45]. Recent evidence indicates that Lewy pathology can propagate trans-neuronally from the olfactory bulb to other regions of the brain in a prion-like manner [30, 47]. This further suggests that pathological transformation of α-Syn in the peripheral olfactory system could be a critical mechanism for PD onset and progression. In line with this hypothesis, olfactory loss often develops in PD patients 4-10 years before the onset of motor symptoms [15-19]. Together these studies strongly suggest that molecular changes in the olfactory system occur early in PD pathogenesis and identifying such molecular changes could reveal biomarkers for early diagnosis of PD.

### (C) Olfactory Cleft Mucus is a Valuable and Easily Accessible Sample Source for Identifying Molecular Changes in the Olfactory Mucosa.

Olfactory cleft mucus, which covers the surface of the epithelium, plays important roles in olfactory tissue homeostasis and odor signal transduction. Cilia of olfactory sensory neurons, where odor detection occurs, are immersed in this mucus [48, 49]. Olfactory mucus contains secreted material from Bowman's glands and sustentacular (i.e., supporting) cells. In addition, olfactory sensory neurons that degenerate, due to cell turnover, exposure to toxic chemicals, or disease processes, release cellular contents into their local environment, which may blend together with the mucus [50]. The olfactory mucus is much more accessible than brain-derived targets, including cerebrospinal fluid (CSF). However, few studies have used olfactory mucus to identify biomarkers for PD.

We use a minimally invasive method to collect human olfactory mucus for biomarker identification. We recently established a method for collecting human olfactory cleft mucus [51]. For this project, we further simplify the method by using nasal swabs to collect olfactory mucus. Olfactory mucus is superior to nasal lavage (a commonly used sampling method for nasal tissues) for biomarker assays, because it is an undiluted, unaltered biofluid that should accurately reflect the microenvironment of the olfactory mucosa. Using shotgun proteomics approach and multiplex immunoassays, we have identified thousands of proteins, including multiple cytokines and growth factors, in the mucus [51, 52]. Furthermore, we discovered that the mucus protein composition undergoes substantial changes during aging, including significant increases in inflammatory biomarkers (e.g., S100A9, S100A8, and monocyte chemoattractant protein-1 [MCP-1, also called CCL-2]) and decreases in anti-oxidative stress proteins (e.g., DJ-1 and glutathione S-transferase P) [51, 52]. As mentioned above, both inflammation and oxidative stress are implicated in PD pathogenesis. Aging is also a dominant risk factor for PD. Thus, these studies demonstrated that olfactory cleft mucus is a valuable and easily accessible sample source for identifying molecular changes in the olfactory mucosa and a promising peripheral source for identifying PD biomarkers.

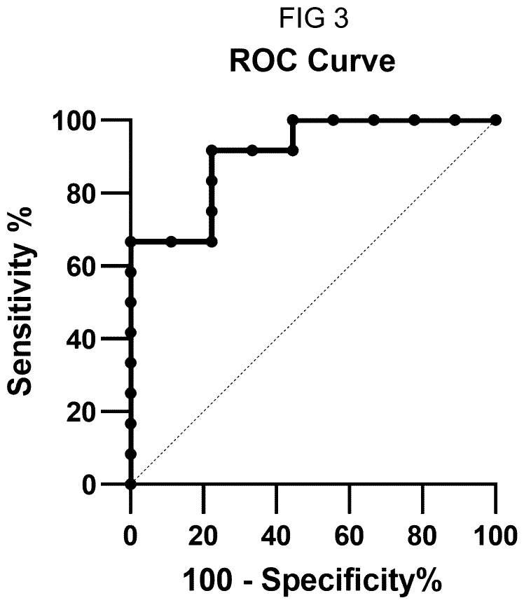

We further investigate a novel combination of protein targets in the olfactory mucus for biomarker development and combine molecular analyses with a machine learning approach to optimize biomarkers for PD. Molecular studies of the peripheral olfactory tissue are more commonly done by RNA analyses. However, such analyses require solid tissue biopsy, which is a much more invasive approach. As described above, our proteomics studies showed that olfactory mucus is a rich protein source for assessing molecular changes in the olfactory mucosa. Based on these large-scale proteomic analyses and our preliminary data, we have selected a novel combination of potential targets for biomarker identification. Our pilot study with a small cohort of PD patients and controls showed that the ratio of two well characterized PD-associated proteins, α-Syn and PD-1, can identify PD patients with high sensitivity and specificity (FIG. 3). For this project, we further validate this protein ratio as a potential PD biomarker. Furthermore, PD is heterogeneous in clinical presentation and disease trajectory, suggesting that combining biomarkers that reflect distinct pathways may greatly enhance the accuracy of PD diagnosis. Thus, we have selected 15-20 candidate proteins from olfactory mucus and use a machine learning approach to identify the best combination of biomarkers for PD (i.e., an algorithm). Our team has demonstrated expertise in machine learning for this approach.

### Example 2. Strategy

Our studies focus on developing a minimally invasive and affordable olfactory biomarker test with strong potential to identify early Parkinson's Disease (PD). Olfactory impairment is present in up to 90% of early-stage PD patients, often developing before the onset of motor symptoms by 4-10 years [15-19]. Among several pre-motor clinical indicators of PD, including olfactory loss, sleep disturbances, and constipation, olfactory loss is the most sensitive predictor of PD [20]. Yet, olfactory impairment has numerous etiologies, including respiratory viral infections, chronic rhinosinusitis, and normal aging [21-24]; so measuring olfactory loss by itself lacks specificity for PD [23]. One way to overcome this hurdle is by developing molecular biomarkers that are directly connected to the underlying mechanisms of PD, therefore distinguishing PD-associated olfactory loss from other types of losses to increase specificity.

Based on our recent proteomics studies of the olfactory cleft mucus, we have selected 15-20 potential biomarkers for PD, including several well-known PD causal proteins, such as α-Synuclein (α-Syn) and DJ-1. We use nasal swabs to collect olfactory mucus, a biofluid covering the surface of the olfactory sensory neurons. We use Luminex-based immunoassays to measure the levels of the proposed biomarkers and use machine learning approach to identify the best algorithm to distinguish PD patients from controls. Our preliminary data showed that the simple ratio of α-Syn/DJ-1 in the olfactory mucus can identify PD patients with high sensitivity and specificity (FIGS. 1C and 3). FIG. 3 shows ROC curve of α-Syn/DJ-1 ratio in olfactory mucus; this ratio shows high biomarker potential for PD. AUC=0.907. p=0.0018. Through the support of the program, we recruit more patients and controls to the study and further optimize the performance of the biomarker test.

The test kit is a simple, safe, and affordable test for PD that can be used as (1) a screening test to select patients who may need a more expensive or invasive PD test for diagnosis, such as DaTscan and RT-QuIC, (2) a test to add specificity for early diagnosis of PD, (3) a companion diagnostic test to select PD patients who would benefit from earlier diagnosis and treatment in clinical trials of pipeline therapeutics, and (4) a universal screening test to all adults over the age of 45 to identify individuals with prodromal PD. This test benefits patients and reduces their health care cost by detecting PD years before motor deficits and creating the opportunity for early intervention and reducing symptoms and patient suffering.

### Example 3. Preliminary Data

### (A) Olfactory α-Syn and DJ-1 as PD Biomarkers.

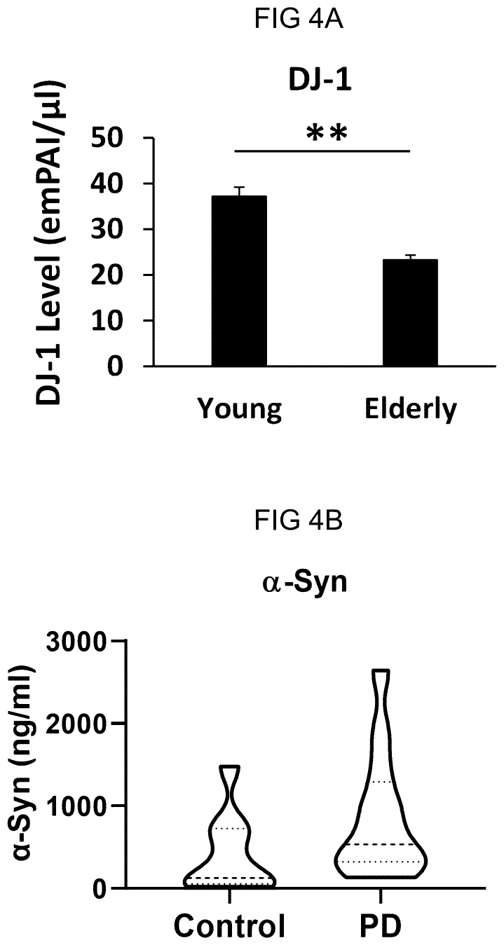

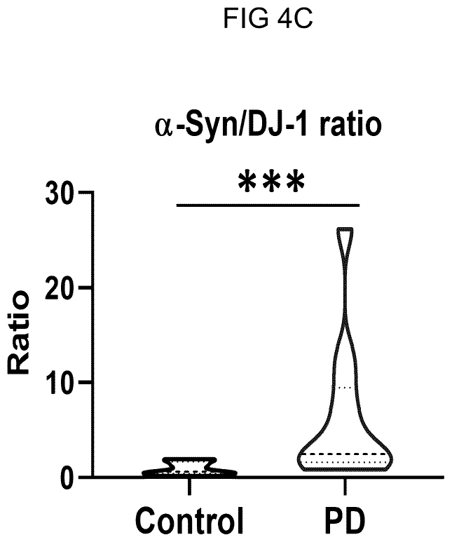

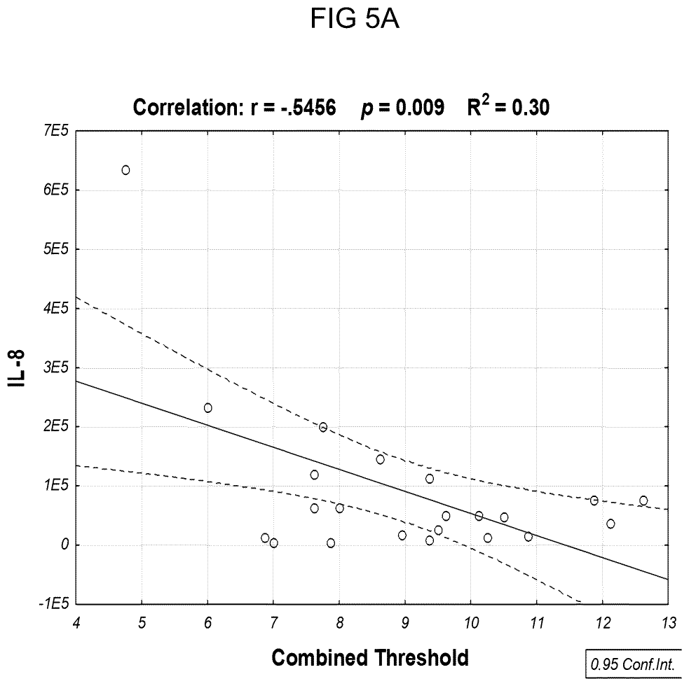

We recently conducted a shotgun proteomics study of human olfactory mucus and identified several thousand proteins in the mucus [51]. We found that DJ-1, a well-known PD-associated protein, is enriched in olfactory mucus compared to anterior nasal mucus [51]. In addition, searching published single-cell RNA sequencing (scRNA-Seq) data sets of human olfactory epithelium, we found that DJ-1 mRNA is expressed in olfactory sensory neurons [54]. DJ-1 has antioxidant activity [55] and can prevent α-Syn from forming aggregates through a number of mechanisms [56-59], including direct interactions with α-Syn [56, 60]. We found that DJ-1 levels in the olfactory mucus decline significantly during aging (FIG. 4A). FIG. 4A shows Levels of DJ-1 in olfactory mucus decline in elderly subjects. Olfactory mucus proteins were analyzed by LC-MS/MS. N=12 for each group. Young: 21-40 years old. Elderly: 65-80 years old. **p=0.004. Because loss-of-function mutations of DJ-1 result in early-onset familial PD [61], reduced levels of DJ-1 during aging may increase the probability of α-Syn pathology in olfactory neurons.

As a follow-up study, we collected olfactory mucus from a cohort of patients (N=12) diagnosed with PD in concordance with UK Brain Bank Parkinson's Disease Diagnostic Criteria and age-matched controls (N=9). We performed Luminex-based multiplex immunoassays to measure levels of three PD-related proteins, α-Syn, DJ-1, and UCHL-1. Compared to the shotgun proteomics approach (LC-MS/MS) for protein identification, Luminex-based immunoassays are faster and more sensitive for less abundant proteins and thus more practical as biomarker assays. We used a commercially available kit, Human Neuroscience Magnetic Bead Panel (Millipore). All of the analyzed proteins were detected in the olfactory mucus.

We found that α-Syn levels were increased in the olfactory mucus of PD patients but did not reach significance (FIG. 4B; control mean=409.5±163.5; PD mean=855.2±222.0; p=0.096). FIG. 4B shows levels of α-Syn are increased in the olfactory mucus of PD patients; N=12 for PD patients; N=9 for age-matched controls; and p=0.096 (Mann-Whitney test, two-tailed). In contrast, the ratio of α-Syn to DJ-1 was significantly elevated in PD patients (FIG. 4C; control mean=0.95+0.22; PD mean=6.20±2.13; p=0.0009). FIG. 4C shows the α-Syn/DJ-1 ratio is markedly elevated in olfactory mucus of PD patients; N=12 for PD patients; N=9 for age-matched controls; and ***p=0.0009 (Mann-Whitney test, two-tailed). This significant difference between PD and control groups persists even after excluding the apparent outlier from the PD group (p=0.0016). These findings are consistent with the known roles of α-Syn and DJ-1 in PD pathology (i.e., DJ-1 inhibits α-Syn from forming toxic oligomeric species [56-58]) and support the hypothesis that an imbalance of the α-Syn and DJ-1 pathways contributes to PD pathogenesis. This hypothesis is also supported by published in vitro and in vivo studies [57, 58, 62]. Moreover, published scRNA-Seq data sets of human olfactory epithelium showed that both α-Syn and DJ-1 are expressed in olfactory sensory neurons [54], suggesting the two proteins may interact within these neurons. Our data also showed that the α-Syn/DJ-1 ratio was highly variable among PD patients but not within controls (FIG. 4C). Such variation may reflect underlying disease progression. In the proposed study, we test the effect of disease progression by recruiting early-and advanced-PD patients.

### (B) The α-Syn/DJ-1 Ratio in Olfactory Mucus Shows Strong Biomarker Potential for PD.

To determine if the α-Syn/DJ-1 ratio, α-Syn alone, or DJ-1 alone in the olfactory mucus has biomarker potential for PD, we performed receiver operating characteristic (ROC) curve analysis and selected the cutoff value at which the sum of sensitivity and specificity is maximal (Table 1). Among the three measurements, the ratio of α-Syn to DJ-1 gives the highest AUC value (0.907; FIG. 3) and the lowest p value (0.0018). At a cutoff of 1.506, the sensitivity and specificity of the α-Syn/DJ-1 ratio for distinguishing PD patients from controls were 92% and 78%, respectively. These diagnostic capabilities are comparable to, if not better than, candidate biomarkers found in CSF [63]

TABLE 1

| Biomarker | AUC | P value | Cutoff value | Sensitivity (%) | Specificity (%) |
|---|---|---|---|---|---|
| α-Syn | 0.907 | 0.0018 | 1.506 | 92 | 78 |
| α-Syn | 0.722 | 0.0881 | 129.3 | 100 | 56 |
| DJ-1 | 0.620 | 0.3556 | 199.8 | 75 | 67 |
| MCP-1 | 0.796 | 0.0230 | 16.38 | 92 | 56 |

### (C) Inflammatory Biomarkers in Olfactory Mucus.

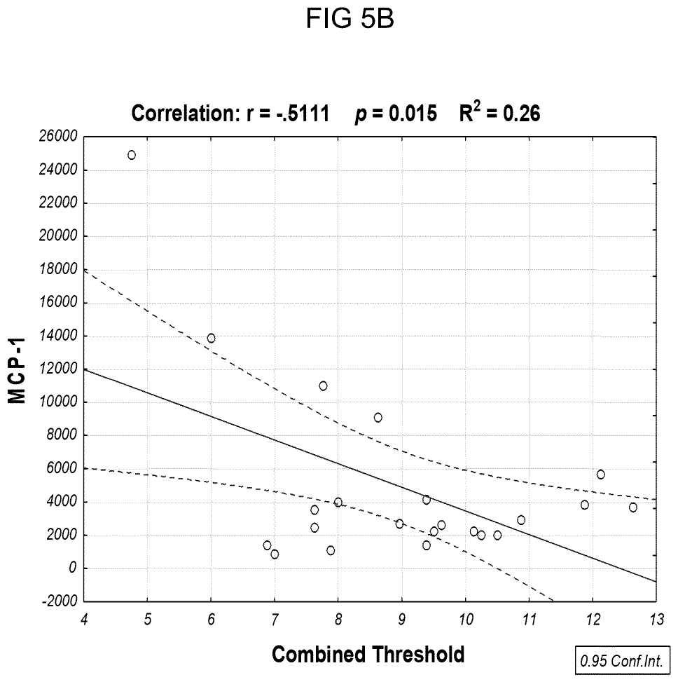

Our recent studies used multiplex immunoassays (20-plex Human Cytokine Panel; Thermo Fisher Scientific) to measure cytokine levels in human olfactory mucus. The levels of several inflammatory cytokines, including interleukin (IL)-8 and MCP-1, in human olfactory mucus is inversely correlated with olfactory performance (FIGS. 5A and 5B). FIG. 5A shows inverse correlation between IL-8 levels and combined olfactory threshold scores for n-butanol and phenylethyl alcohol; correlation coefficients (r) and p values are indicated in each panel. FIG. 5B shows Inverse correlation between MCP-1 levels and combined olfactory threshold scores for n-butanol and phenylethyl alcohol; correlation coefficients (r) and p values are indicated in each panel.

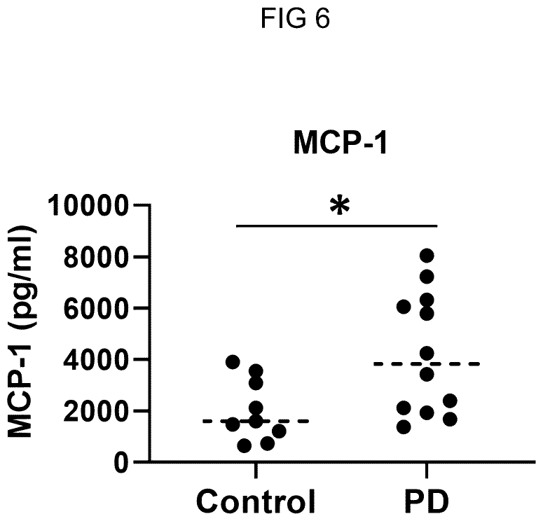

Further, we found that MCP-1 levels in the olfactory mucus of PD patients are significantly higher than in age-matched controls (FIG. 6; control mean=2035±402.7, PD mean=4217±686.1; p=0.023), consistent with impaired olfactory function in PD patients. FIG. 6 shows that levels of MCP-1 are significantly increased in the olfactory mucus of PD patients; N=12 for PD patients; N=9 for age-matched controls; and *p=0.023 (Mann-Whitney test, two-tailed). In agreement with this finding, it has been reported that levels of MCP-1 are increased in other biofluids, such as CSF and blood, of PD patients [36, 64]. It appears that the human olfactory mucosa is under strong inflammatory stress during aging. Both shotgun proteomics and multiplex immunoassays show increased levels of inflammatory biomarkers in olfactory mucus of elderly subjects [51, 52]. The further increased levels of MCP-1 in the olfactory mucus of PD patients are consistent with the role of inflammation and aging in PD pathogenesis [65].

Together, our published studies and preliminary results strongly suggest that molecular changes in the olfactory mucosa are associated with PD pathogenesis; and that protein analysis of olfactory mucus can identify such changes, providing novel biomarkers for PD.

### Example 4. Further Studies

The goal of this project is to develop a simple, safe, minimally invasive, and affordable biomarker test that can be used as a screening test to identify early-stage PD patients, so that patients can be treated early to have a better outcome. With the various disease modifying therapies on the horizon, early screening for PD will be in high demand. However, the two currently available tests for PD, RT-QuIC (SYNTap) and DaTscan; are not broadly available or suited for patient screening due to either invasiveness and/or high cost.

The olfactory system undergoes pathological changes in the earliest stage of PD. The olfactory sensory epithelium is accessible through the nose. Our team has shown that olfactory mucus, a biofluid covering the surface of olfactory sensory neurons, can be collected through minimally invasive methods. More important, we discovered that olfactory mucus contains critical biomarkers directly connected to the underlying pathology of PD, including α-Syn and DJ-1. Furthermore, our preliminary results show that the ratio of α-Syn to DJ-1 in the olfactory mucus is dramatically elevated in PD patients and exhibits strong biomarker potential (AUC=0.907 in ROC analysis, FIG. 3). The above-mentioned results are confirmed in larger cohorts of PD patients and controls, therefore our olfactory biomarker test can offer a simple, safe, and affordable alternative for identifying PD patients.

### (A) Validate Whether the Ratio of α-Syn to DJ-1 in the Olfactory Mucus is a Biomarker for PD.

Alpha-Synuclein (α-Syn) is a major component of Lewy bodies and neurites found in both sporadic and inherited PD, whereas DJ-1 is a redox-dependent molecular chaperone that can prevent α-Syn aggregation. The ratio of α-Syn to DJ-1 in the olfactory mucus is dramatically increased in PD patients compared to controls (FIG. 4C), strongly consistent with the proteins' roles in PD pathogenesis. Therefore, in this study we validate the biomarker potential of α-Syn/DJ-1 ratio in a larger cohort of PD patients and age-matched controls.

### Patient Recruitment and Sample Collection

We recruit 30 PD patients through the Parkinson's Disease Research, Education and Clinical Center (PADRECC) at the Corporal Michael J. Crescenz VA Medical Center (CMCVAMC). To maximize patient recruitment in the short study period, we recruit patients from all stages (all Hoehn and Yahr stages) [66]. Both men and women are included. Because more men than women have PD and PADRECC patients are predominantly male, we further target females by performing patient database search and mail a recruitment letter to all potentially eligible females. In case our cohort is unbalanced in gender, we recruit the same proportions of men and women for the control group. Control subjects should not have neurological disorders, have a normal sense of smell, and have no family history of PD in first-degree relatives. Exclusion criteria for all subjects include active sinonasal infection, chronic rhinosinusitis, and acute respiratory infection (e.g., COVID-19 and flu). A rapid antigen test for COVID-19 will be performed, and positive subjects are not included. Because of the strong effect of COVID-19 on olfaction [67, 68], subjects with a history of COVID-19 infection ware also be excluded. A simple odor identification test will be given to all subjects to assess their olfactory function [69]. Dr. James Morley, MD, PHD (Co-PI), Co-director of the PADRECC and a movement disorders neurologist, supervises patient recruitment. Dr. Hong Wang (PI) and Dr. Pamela Dalton (Investigator) at the Monell Center supervises recruitment of control subjects. We make every effort to match patient and control populations in key demographics (age, sex, race, etc.).

All subjects provide an informed consent prior to participation. Olfactory mucus collection is done by using nasal swabs with soft, flexible, and absorbing tips. A swab are placed close to the olfactory groove between the middle turbinate and superior nasal septum in one nostril. After 10 min, the swab is removed from the nose, and the absorbing tip is cut off, placed in a microfuge tube, and centrifuged at 10,000 rpm for 2 min to collect the olfactory mucus. Mucus is aliquoted, snap-frozen on dry ice, and stored at −80° C. for batch processing. All samples are processed and handled in the same way to minimize variations due handling and storage. Handling and storage is performed under study coordinator supervision.

### Detection of α-Syn and DJ-1 by Multiplex Immunoassays

We use Luminex-based, multiplex immunoassays for protein detection. These multiplex immunoassays are well established, and their detection sensitivities of olfactory mucus proteins are sufficient for this project based on our pilot data. These assays utilize Luminex xMAP technology that uses fluorescently labeled magnetic beads for detection and measurement. Similar to enzyme-linked immunosorbent assays, these multiplex immunoassays use capture and detection antibody pairs to enhance specificity. Data acquisition is done using Luminex instruments at the Human Immunology Core of the University of Pennsylvania. We use the Human Neuroscience Magnetic Bead Panel (Millipore) to detect α-Syn and DJ-1 in the olfactory mucus. The kit provides assay standards and quality control samples for data analysis and quality control and has been used by others for neurodegeneration studies [70, 71]. In our pilot experiments, α-Syn and DJ-1 in the olfactory mucus are easily detectable using this immunoassay kit (FIGS. 4B and 4C).

### Data Analysis

We perform statistical analyses to determine the following: (1) use unpaired t-tests to determine if the ratio of α-Syn to DJ-1 are significantly different between controls and PD patients; and (2) perform ROC analyses to determine if the ratio of α-Syn to DJ-I have biomarker potential to distinguish controls from PD patients.

### Outcomes

Genetic studies have shown that multiplication, such as duplication and triplication, of the wild-type α-Syn gene results in early-onset familial PD [27, 28]. This gene dosage effect suggests that higher levels of α-Syn are a risk factor for PD pathogenesis. Our preliminary results showed that levels of α-Syn in olfactory mucus trended higher in PD patients (FIG. 4B), consistent with published histological evidence indicating that α-Syn pathology initiates in the olfactory system [43]. We anticipate that with a larger cohort of participants we observe a statistically significant difference in levels of α-Syn between PD patients and controls. However, we acknowledge that the utility of total α-Syn levels alone as a PD biomarker is controversial [72]. Our preliminary results showed that the ratio of α-Syn to DJ-1 in olfactory mucus is markedly elevated in PD patients (FIG. 4C) and is a better predicator of PD than α-Syn level alone (FIG. 3, Table 1). These data indicate that imbalance of α-Syn and DJ-1 in olfactory mucus is strongly associated with PD and consistent with the known roles of α-Syn and DJ-1 in PD pathogenesis. We anticipate that similar results are obtained with the larger cohort in the proposed study. The immunoassays we propose to use for measuring α-Syn and DJ-1 are sensitive and reproducible. Our preliminary studies have demonstrated their feasibility. We expect that the ratio of α-Syn to DJ-1 in olfactory mucus can achieve AUC=0.9 in ROC analysis.

### (B) Develop an Algorithm to Optimize Olfactory Biomarkers for PD.

PD is a complex disease with heterogeneous disease trajectory among individual patients. Multiple biological pathways are implicated in PD pathogenesis. Therefore, a biomarker panel, including candidates from major pathways implicated in PD, may offer higher sensitivity and specificity than Syn/DJ-1 ratio. Based on this consideration, we have selected ˜15 candidates to be included in a biomarker panel. We use machine learning approaches to build an algorithm to optimize olfactory biomarkers for PD.

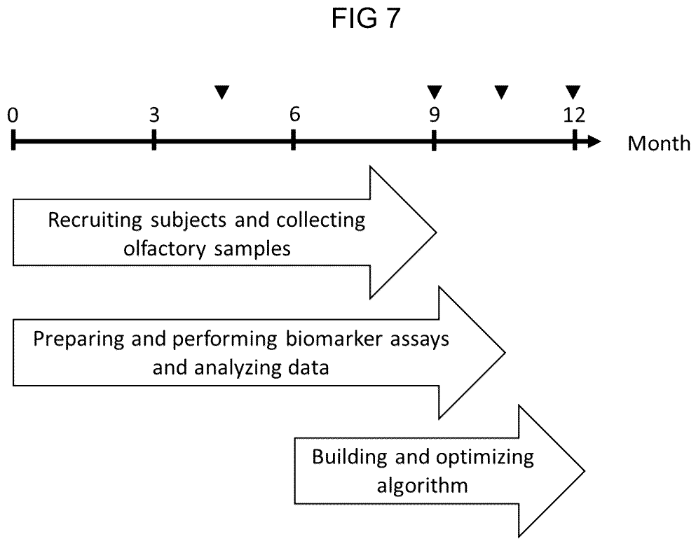

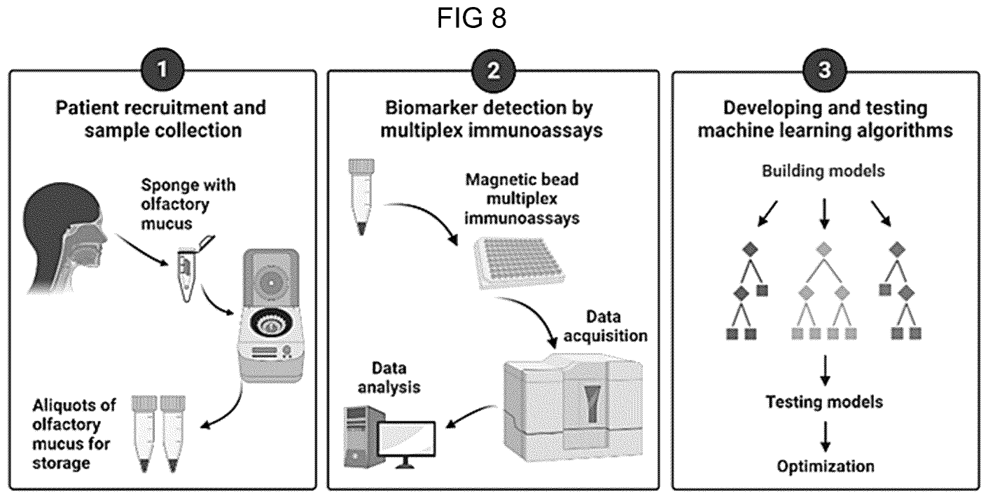

The timeline of the project is shown in FIG. 7. FIG. 8 shows schematic representation of sample collection, data acquisition, and algorithm optimization. The two aspects of the project as described in EXAMPLE 4: (A) and (B) share patient samples and are carried out in parallel. Milestones of the project are the following: (1) By 4.5 months, we recruit and collect olfactory mucus samples from 15 PD patients and 15-matched controls. (2) By 9 months, we recruit a total of 30 PD patients and 30 controls; and finish sample collection from all 60 subjects. (3) By 10.5 months, we finish all proposed biomarker assays and data acquisition. (4) By 12 months, we finish analyzing data and building and optimizing biomarker algorithms.

### Multiplex Immunoassays for an Olfactory Biomarker Panel

Inflammation goes hand in hand with oxidative stress, protein misfolding, and neurodegeneration. Although the exact role of inflammation in PD remains unclear, the association between inflammation and PD is well accepted [35, 73, 74]. Our proteomics studies have shown that levels of various inflammatory biomarkers in human olfactory mucus are significantly increased in elderly subjects, suggesting that the olfactory mucosa is under substantial inflammatory stress in old age. In fact, S100A9 and S100A8, two inflammatory biomarkers [75], are the two most increased proteins (increased 16.4- and 15.3-fold, respectively) in olfactory mucus of elderly subjects in our data set [51]. S100A8 and S100A9 are expressed in neutrophils and monocytes and are released from cells during inflammation [75]. Levels of MCP-1, an inflammatory chemokine, are also significantly increased in the olfactory mucus of elderly subjects [52]. Moreover, our preliminary results showed that levels of MCP-1 were further elevated in the olfactory mucus of PD patients compared to age-matched controls (FIG. 6). Others have reported that MCP-1 levels are increased in blood, tears, and CSF of PD patients [36, 64, 76]. Additionally, levels of MCP-1 in CSF positively correlate with PD progression [36]. Although it is expected that inflammatory biomarkers are noisy indicators because of their broad response to an array of diseases, the fact that some inflammatory biomarkers are significantly associated with PD suggests that these biomarkers can be useful, especially as components of biomarker panels [36, 77].

Besides inflammation, oxidative stress also contributes to PD. Glutathione is an endogenous antioxidant, and loss of glutathione has been implicated in PD [78]. We have detected a significant reduction of glutathione S-transferase P in the olfactory mucus of elderly subjects, although its association with PD is unclear.

We have selected ˜15 potential biomarkers that we examine using Luminex-based multiplex immunoassays. We use custom-selected multiplex Panels (Thermo Fisher Scientific and Millipore) These multiplex immunoassay panels are well established and have been used in studies of neurological disorders (including PD), cancer, and others [79-84]. Data from these analyses are used to build machine learning models (see below) to develop a PD biomarker algorithm.

### Developing and Testing Algorithms by Machine Learning

For the machine learning approach, data are divided into a training set from which the model learns and a test set on which the performance of the model is measured. Models can memorize the training data (“over-fitting”), so it is critical to use data the models have never seen (test set) to measure generalizable performance; we reserve 20% of the data to form a test set. Selecting the best algorithm for a problem is often an empirical process; we apply five algorithms that have been successfully applied to similar problems: regularized linear models, support vector machine, random forest, extreme gradient boosting, and neural networks. It is straightforward to train and test these models in the R program using the caret package, as we demonstrated previously [53]. As a negative control, we train the models on data that are randomly shuffled to remove correlations between biomarkers and phenotypes. This determines the chance level of prediction.

### Outcomes

PD shows considerable heterogeneity in clinical presentation and disease progression, which contributes to the difficulty of developing highly predictive biomarkers for PD [12, 85]. One strategy to overcome this difficulty is to use a biomarker panel and optimize the predictive algorithms. Building and testing machine learning models fit ideally with this strategy. We expect that our selected 15 analytes combined with the machine learning approach identifies a superior combination of olfactory biomarkers for PD and surpass the performance of α-Syn/DJ-1 ratio (i.e., AUC>0.9).

### REFERENCES

1. Wooten, G.F., Neurochemistry and neuropharmacology of Parkinson's disease, in Movement disorders: neurologic principles and practice, R.L. Watts and W. Koller, Editors. 1997, McGraw-Hill: New York. p. 153-160.
2. Yang, W., et al., Current and projected future economic burden of Parkinson's disease in the U.S. NPJ Parkinsons Dis, 2020. 6: p. 15.
3. Marras, C., et al., Prevalence of Parkinson's disease across North America. NPJ Parkinsons Dis, 2018. 4: p. 21.
4. Van Den Eeden, S.K., et al., Incidence of Parkinson's disease: variation by age, gender, and race/ethnicity. Am J Epidemiol, 2003. 157(11): p. 1015-22.
5. LewinGroup, Economic Burden and Future Impact of Parkinson's Disease. michaeljfox.org/sites/default/files/media/document/2019%20Parkinson%27s%20Economic%20Burden%20Study%20-%20FINAL.pdf, 2019.
6. Driver, J.A., et al., Incidence and remaining lifetime risk of Parkinson disease in advanced age. Neurology, 2009. 72(5): p. 432-8.
7. Martinez-Martin, P., et al., The long-term direct and indirect economic burden among Parkinson's disease caregivers in the United States. Mov Disord, 2019. 34(2): p. 236-245.
8. Ferrazzoli, D., et al., Dopamine Replacement Therapy, Learning and Reward Prediction in Parkinson's Disease: Implications for Rehabilitation. Front Behav Neurosci, 2016. 10: p. 121.
9. Elkouzi, A., et al., Emerging therapies in Parkinson disease—repurposed drugs and new approaches. Nat Rev Neurol, 2019. 15(4): p. 204-223.
10. Fields, C.R., N. Bengoa-Vergniory, and R. Wade-Martins, Targeting Alpha-Synuclein as a Therapy for Parkinson's Disease. Front Mol Neurosci, 2019. 12: p. 299.
11. Kalia, L.V., S.K. Kalia, and A.E. Lang, Disease-modifying strategies for Parkinson's disease. Mov Disord, 2015. 30(11): p. 1442-50.
12. Tropea, T.F. and A.S. Chen-Plotkin, Unlocking the mystery of biomarkers: A brief introduction, challenges and opportunities in Parkinson Disease. Parkinsonism Relat Disord, 2018. 46 Suppl 1: p. S15-S18.
13. Stephenson, D., et al., The Qualification of an Enrichment Biomarker for Clinical Trials Targeting Early-stages of Parkinson's Disease. J Parkinsons Dis, 2019. 9(3): p. 553-563.
14. Crotty, G.F. and M.A. Schwarzschild, Chasing Protection in Parkinson's Disease: Does Exercise Reduce Risk and Progression? Front Aging Neurosci, 2020. 12: p. 186.
15. Doty, R.L., Olfactory dysfunction in Parkinson disease. Nat Rev Neurol, 2012. 8(6): p. 329-39.
16. Ross, G.W., et al., Association of olfactory dysfunction with risk for future Parkinson's disease. Ann Neurol, 2008. 63(2): p. 167-73.
17. Mantri, S., J.E. Duda, and J.F. Morley, Early and Accurate Identification of Parkinson Disease Among US Veterans. Fed Pract, 2019. 36(Suppl 4): p. S18-S23.
18. Morley, J.F., et al., Optimizing olfactory testing for the diagnosis of Parkinson's disease: item analysis of the university of Pennsylvania smell identification test. NPJ Parkinsons Dis, 2018. 4: p. 2.
19. Fullard, M.E., J.F. Morley, and J.E. Duda, Olfactory Dysfunction as an Early Biomarker in Parkinson's Disease. Neurosci Bull, 2017. 33(5): p. 515-525.
20. Postuma, R.B., et al., Identifying prodromal Parkinson's disease: pre-motor disorders in Parkinson's disease. Mov Disord, 2012. 27(5): p. 617-26.
21. Schiffman, S.S., Taste and smell in disease (second of two parts). N Engl J Med, 1983. 308(22): p. 1337-43.
22. Cowart, B.J., et al., Clinical disorders of smell and taste. Occup Med, 1997. 12(3): p. 465-83.
23. Murphy, C., R.L. Doty, and H.J. Duncan, Clinical Disorders of Olfaction, in Handbook of Olfaction and Gustation, R.L. Doty, Editor. 2003, Marcel Dekker: New York. p. 461-478.
24. Bromley, S.M., Smell and taste disorders: a primary care approach. Am Fam Physician, 2000. 61: p. 427-438.
25. Polymeropoulos, M.H., et al., Mutation in the alpha-synuclein gene identified in families with Parkinson's disease. Science, 1997. 276(5321): p. 2045-7.
26. Kruger, R., et al., Ala30Pro mutation in the gene encoding alpha-synuclein in Parkinson's disease. Nat Genet, 1998. 18(2): p. 106-8.
27. Singleton, A.B., et al., alpha-Synuclein locus triplication causes Parkinson's disease. Science, 2003. 302(5646): p. 841.
28. Chartier-Harlin, M.C., et al., Alpha-synuclein locus duplication as a cause of familial Parkinson's disease. Lancet, 2004. 364(9440): p. 1167-9.
29. Bengoa-Vergniory, N., et al., Alpha-synuclein oligomers: a new hope. Acta Neuropathol, 2017. 134(6): p. 819-838.
30. Rey, N.L., et al., Widespread transneuronal propagation of alpha-synucleinopathy triggered in olfactory bulb mimics prodromal Parkinson's disease. J Exp Med, 2016. 213(9): p. 1759-78.
31. Tolosa, E., et al., LRRK2 in Parkinson disease: challenges of clinical trials. Nat Rev Neurol, 2020. 16(2): p. 97-107.
32. Klein, C. and A. Westenberger, Genetics of Parkinson's disease. Cold Spring Harb Perspect Med, 2012. 2(1): p. a008888.
33. Chang, D., et al., A meta-analysis of genome-wide association studies identifies 17 new Parkinson's disease risk loci. Nat Genet, 2017. 49(10): p. 1511-1516.
34. Corces, M.R., et al., Single-cell epigenomic analyses implicate candidate causal variants at inherited risk loci for Alzheimer's and Parkinson's diseases. Nat Genet, 2020. 52(11): p. 1158-1168.
35. Mosley, R.L., et al., Inflammation and adaptive immunity in Parkinson's disease. Cold Spring Harb Perspect Med, 2012. 2(1): p. a009381.
36. Santaella, A., et al., Inflammation biomarker discovery in Parkinson's disease and atypical parkinsonisms. BMC Neurol, 2020. 20(1): p. 26.
37. Hawkes, C.H., K. Del Tredici, and H. Braak, Parkinson's disease: a dual-hit hypothesis. Neuropathol Appl Neurobiol, 2007. 33(6): p. 599-614.
38. Doty, R.L., Olfaction in Parkinson's disease. Parkinsonism Relat Disord, 2007. 13 Suppl 3: p. S225-8.
39. Dickson, D.W., Parkinson's disease and parkinsonism: neuropathology. Cold Spring Harb Perspect Med, 2012. 2(8).
40. Braak, H., et al., Gastric alpha-synuclein immunoreactive inclusions in Meissner's and Auerbach's plexuses in cases staged for Parkinson's disease-related brain pathology. Neurosci Lett, 2006. 396(1): p. 67-72.
41. Lebouvier, T., et al., Pathological lesions in colonic biopsies during Parkinson's disease. Gut, 2008. 57(12): p. 1741-3.
42. Shannon, K.M., et al., Alpha-synuclein in colonic submucosa in early untreated Parkinson's disease. Mov Disord, 2012. 27(6): p. 709-15.
43. Del Tredici, K., et al., Where does parkinson disease pathology begin in the brain? J Neuropathol Exp Neurol, 2002. 61(5): p. 413-26.
44. Braak, H., et al., Staging of brain pathology related to sporadic Parkinson's disease. Neurobiol Aging, 2003. 24(2): p. 197-211.
45. Saito, Y., et al., Lewy body pathology involves the olfactory cells in Parkinson's disease and related disorders. Mov Disord, 2016. 31(1): p. 135-8.
46. Funabe, S., et al., Neuropathologic analysis of Lewy-related alpha-synucleinopathy in olfactory mucosa. Neuropathology, 2013. 33(1): p. 47-58.
47. Rey, N.L., D.W. Wesson, and P. Brundin, The olfactory bulb as the entry site for prion-like propagation in neurodegenerative diseases. Neurobiol Dis, 2018. 109(Pt B): p. 226-248.
48. Getchell, M.L. and T.V. Getchell, Fine structural aspects of secretion and extrinsic innervation in the olfactory mucosa. Microsc Res Tech, 1992. 23(2): p. 111-27.
49. Choi, R. and B.J. Goldstein, Olfactory epithelium: Cells, clinical disorders, and insights from an adult stem cell niche. Laryngoscope Investig Otolaryngol, 2018. 3(1): p. 35-42.
50. Nikula, K.J. and J.L. Lewis, Olfactory mucosal lesions in F344 rats following inhalation exposure to pyridine at threshold limit value concentrations. Fundam Appl Toxicol, 1994. 23(4): p. 510-7.
51. Yoshikawa, K., et al., The human olfactory cleft mucus proteome and its age-related changes. Sci Rep, 2018. 8(1): p. 17170.
52. Wang, H., et al., Cytokine profile in human olfactory cleft mucus and associated changes in olfactory function. bioRxiv, 2018 332395: p. doi: https://doi.org/10.1101/332395.
53. Keller, A., et al., Predicting human olfactory perception from chemical features of odor molecules. Science, 2017. 355(6327): p. 820-826.
54. Durante, M.A., et al., Single-cell analysis of olfactory neurogenesis and differentiation in adult humans. Nat Neurosci, 2020. 23(3): p. 323-326.
55. Choi, J., et al., Oxidative damage of DJ-1 is linked to sporadic Parkinson and Alzheimer diseases. J Biol Chem, 2006. 281(16): p. 10816-24.
56. Zhu, M., H.P. S, and S. Han, DJ-1, a Parkinson's disease related protein, aggregates under denaturing conditions and co-aggregates with alpha-synuclein through hydrophobic interaction. Biochim Biophys Acta Gen Subj, 2017. 1861(7): p. 1759-1769.
57. Xu, C.Y., et al., DJ-1 Inhibits alpha-Synuclein Aggregation by Regulating Chaperone-Mediated Autophagy. Front Aging Neurosci, 2017. 9: p. 308.
58. Zhou, W. and C.R. Freed, DJ-1 up-regulates glutathione synthesis during oxidative stress and inhibits A53T alpha-synuclein toxicity. J Biol Chem, 2005. 280(52): p. 43150-8.
59. Shendelman, S., et al., DJ-1 is a redox-dependent molecular chaperone that inhibits alpha-synuclein aggregate formation. PLOS Biol, 2004. 2(11): p. e362.
60. Zondler, L., et al., DJ-1 interactions with alpha-synuclein attenuate aggregation and cellular toxicity in models of Parkinson's disease. Cell Death Dis, 2014. 5: p. e1350.
61. Bonifati, V., et al., Mutations in the DJ-1 gene associated with autosomal recessive early-onset parkinsonism. Science, 2003. 299(5604): p. 256-9.
62. Zhou, W., et al., Phenylbutyrate up-regulates the DJ-1 protein and protects neurons in cell culture and in animal models of Parkinson disease. J Biol Chem, 2011. 286(17): p. 14941-51.
63. Hong, Z., et al., DJ-1 and alpha-synuclein in human cerebrospinal fluid as biomarkers of Parkinson's disease. Brain, 2010. 133(Pt 3): p. 713-26.
64. Grozdanov, V., et al., Inflammatory dysregulation of blood monocytes in Parkinson's disease patients. Acta Neuropathol, 2014. 128(5): p. 651-63.
65. Joshi, N. and S. Singh, Updates on immunity and inflammation in Parkinson disease pathology. J Neurosci Res, 2018. 96(3): p. 379-390.
66. Mantri, S., et al., Comparing self-reported and objective monitoring of physical activity in Parkinson disease. Parkinsonism Relat Disord, 2019. 67: p. 56-59.
67. Cooper, K.W., et al., COVID-19 and the Chemical Senses: Supporting Players Take Center Stage. Neuron, 2020. 107(2): p. 219-233.
68. Parma, V., et al., More Than Smell-COVID-19 Is Associated With Severe Impairment of Smell, Taste, and Chemesthesis. Chem Senses, 2020. 45(7): p. 609-622.
69. Dalton, P., et al., Olfactory assessment using the NIH Toolbox. Neurology, 2013. 80(11 Suppl 3): p. S32-6.
70. Fanning, J.P., et al., Neuron-Specific Enolase and Matrix Metalloproteinase 9 Signal Perioperative Silent Brain Infarction During or After Transcatheter Aortic Valve Implantation. Am J Cardiol, 2019. 123(3): p. 434-439.
71. Trapecar, M., et al., Human physiomimetic model integrating microphysiological systems of the gut, liver, and brain for studies of neurodegenerative diseases. Sci Adv, 2021. 7(5).
72. Mantri, S., J.F. Morley, and A.D. Siderowf, The importance of preclinical diagnostics in Parkinson disease. Parkinsonism Relat Disord, 2019. 64: p. 20-28.
73. Perry, V.H., Innate inflammation in Parkinson's disease. Cold Spring Harb Perspect Med, 2012. 2(9): p. a009373.
74. Kannarkat, G.T., J.M. Boss, and M.G. Tansey, The role of innate and adaptive immunity in Parkinson's disease. J Parkinsons Dis, 2013. 3(4): p. 493-514.
75. Wang, S., et al., S100A8/A9 in Inflammation. Front Immunol, 2018. 9: p. 1298.
76. Hamm-Alvarez, S.F., et al., Levels of oligomeric alpha-Synuclein in reflex tears distinguish Parkinson's disease patients from healthy controls. Biomark Med, 2019. 13(17): p. 1447-1457.
77. Brockmann, K., et al., Inflammatory profile discriminates clinical subtypes in LRRK2-associated Parkinson's disease. Eur J Neurol, 2017. 24(2): p. 427-e6.
78. Martin, H.L. and P. Teismann, Glutathione—a review on its role and significance in Parkinson's disease. FASEB J, 2009. 23(10): p. 3263-72.
79. Chang, D.H., et al., Sustained expansion of NKT cells and antigen-specific T cells after injection of alpha-galactosyl-ceramide loaded mature dendritic cells in cancer patients. J Exp Med, 2005. 201(9): p. 1503-17.
80. Cook, D.B., et al., Multiplexing protein and gene level measurements on a single Luminex platform. Methods, 2019. 158: p. 27-32.
81. Hofer, L.S., et al., Distinct serum and cerebrospinal fluid cytokine and chemokine profiles in autoantibody-associated demyelinating diseases. Mult Scler J Exp Transl Clin, 2019. 5(2): p. 2055217319848463.
82. Jung, H.H., et al., Cytokine profiling in serum-derived exosomes isolated by different methods. Sci Rep, 2020. 10(1): p. 14069.
83. Lin, C.H., et al., Altered gut microbiota and inflammatory cytokine responses in patients with Parkinson's disease. J Neuroinflammation, 2019. 16(1): p. 129.
84. Haenseler, W., et al., Excess alpha-synuclein compromises phagocytosis in iPSC-derived macrophages. Sci Rep, 2017. 7(1): p. 9003.
85. Yilmaz, R., et al., Biomarkers of Parkinson's disease: 20 years later. J Neural Transm (Vienna), 2019. 126(7): p. 803-813.
86. Reichmann, H., Clinical criteria for the diagnosis of Parkinson's disease. Neurodegener Dis, 2010. 7(5): p. 284-90.
87. Postuma, R.B., et al., MDS clinical diagnostic criteria for Parkinson's disease. Mov Disord, 2015. 30(12): p. 1591-601.
88. Hughes, A.J., S.E. Daniel, and A.J. Lees, Improved accuracy of clinical diagnosis of Lewy body Parkinson's disease. Neurology, 2001. 57(8): p. 1497-9.
89. Marshall, V.L., et al., Parkinson's disease is overdiagnosed clinically at baseline in diagnostically uncertain cases: a 3-year European multicenter study with repeat [123I]FP-CIT SPECT. Mov Disord, 2009. 24(4): p. 500-8.
90. Jennings, D., et al., Conversion to Parkinson Disease in the PARS Hyposmic and Dopamine Transporter-Deficit Prodromal Cohort. JAMA Neurol, 2017. 74(8): p. 933-940.
91. Bega, D., et al., Clinical utility of DaTscan in patients with suspected Parkinsonian syndrome: a systematic review and meta-analysis. NPJ Parkinsons Dis, 2021. 7(1): p. 43.
92. Dong, T.T. and K. Satoh, The Latest Research on RT-QuIC Assays-A Literature Review. Pathogens, 2021. 10(3).
93. Fairfoul, G., et al., Alpha-synuclein RT-QuIC in the CSF of patients with alpha-synucleinopathies. Ann Clin Transl Neurol, 2016. 3(10): p. 812-818.
94. Saijo, E., et al., Ultrasensitive RT-QuIC Seed Amplification Assays for Disease-Associated Tau, alpha-Synuclein, and Prion Aggregates. Methods Mol Biol, 2019. 1873: p. 19-37.
95. Han, M., et al., Diagnosis of Parkinson's disease based on disease-specific autoantibody profiles in human sera. PLOS One, 2012. 7(2): p. e32383.
96. Khoo, S.K., et al., Plasma-based circulating MicroRNA biomarkers for Parkinson's disease. J Parkinsons Dis, 2012. 2(4): p. 321-31.
97. Donadio, V., et al., Skin nerve alpha-synuclein deposits: a biomarker for idiopathic Parkinson disease. Neurology, 2014. 82(15): p. 1362-9.
98. Bougea, A., C. Koros, and L. Stefanis, Salivary alpha-synuclein as a biomarker for Parkinson's disease: a systematic review. J Neural Transm (Vienna), 2019. 126(11): p. 1373-1382.
99. Visanji, N.P., et al., The Systemic Synuclein Sampling Study: toward a biomarker for Parkinson's disease. Biomark Med, 2017. 11(4): p. 359-368.

All documents cited in this specification are incorporated herein by reference. U.S. Provisional Patent Application No. 63/271, 989, filed Oct. 26, 2021 is incorporated herein by reference in its entirety. While the invention has been described with reference to particular embodiments, it will be appreciated that modifications can be made without departing from the spirit of the invention. Such modifications are intended to fall within the scope of the appended claims.

1. A composition for diagnosing the existence or evaluating progression of Parkinson's Disease (PD) in a subject in need thereof, wherein the composition comprises at least one ligand, wherein the at least one ligand is specific for and binds to a protein biomarker in a sample collected from a subject, wherein the protein biomarker is selected from BDNF, EGF, EOTAXIN, FGF2, HGF, IFNa, IFNg, IL1RA, IL10, IL12P40, IL13, IL6, IL8/CXCL8, IP10, MCP-1/CCL2, MIG, MIP1A, MIP1B, RANTES, VEGFa, alpha-Synuclein (α-Syn), Park7/DJ-1 (DJ-1), Park5/UCHL1, LRRK2, Mapt (i.e., tau), Parkin, PINK1, GBA, S100A8, S100A9, Histone H4, Transthyretin, hemoglobin subunit delta, hemoglobin subunit alpha, hemoglobin subunit beta, Glutathione S-transferase P, Cystatin-A, PEBP1, HSPA2, TUBB3, DNAL1, and Krt14, and wherein the ligand is for measuring the expression level of the protein biomarker.

2. The composition according to claim 1, wherein the composition comprises multiple ligands, each ligand being specific for and binding to a different protein biomarker selected from BDNF, EGF, EOTAXIN, FGF2, HGF, IFNa, IFNg, IL1RA, IL10, IL12P40, IL13, IL6, IL8/CXCL8, IP10, MCP-1/CCL2, MIG, MIP1A, MIP1B, RANTES, VEGFa, alpha-Synuclein (α-Syn), Park7/DJ-1 (DJ-1), Park5/UCHL1, LRRK2, Mapt (i.e., tau), Parkin, PINK1, GBA, S100A8, S100A9, Histone H4, Transthyretin, hemoglobin subunit delta, hemoglobin subunit alpha, hemoglobin subunit beta, Glutathione S-transferase P, Cystatin-A, PEBP1, HSPA2, TUBB3, DNAL1, and Krt14.

3. (canceled)

4. The composition according to claim 1, wherein at least one ligand is attached to a detectable label.

5. The composition according to claim 4, wherein each of the ligands is attached to a different detectable label.

6. The composition according to claim 1, wherein the protein biomarker is α-Syn.

7. The composition according to claim 1, wherein the protein biomarker is DJ-1.

8. The composition according to claim 1, wherein the protein biomarker is MCP-1.

9. The composition according to claim 1, wherein the composition comprises ligands specific for and bind to protein biomarkers alpha-synuclein (α-Syn) and DJ-1 for use in calculating the α-Syn/DJ-1 ratio.

10. A kit comprising the composition of claim 1 and an apparatus for sample collection.

11. The kit according to claim 10, wherein said apparatus for sample collection comprises a polypropylene sponge for mucus collection, and a tube for storage of the sponge, optionally wherein the tube contains a reagent which stabilizes the mucus sample.

12. A method of diagnosing existence or evaluating progression of Parkinson's Disease (PD) in a subject, wherein the method comprising:
(i) measuring an expression level of one or more protein biomarker in the subject, wherein the one or more protein biomarker is selected from BDNF, EGF, EOTAXIN, FGF2, HGF, IFNa, IFNg, IL1RA, IL10, IL12P40, IL13, IL6, IL8/CXCL8, IP10, MCP-1/CCL2, MIG, MIP1A, MIP1B, RANTES, VEGFa, alpha-Synuclein (α-Syn), Park7/DJ-1 (DJ-1), Park5/UCHL1, LRRK2, Mapt (tau), Parkin, PINK1, GBA, S100A8, S100A9, Histone H4, Transthyretin, hemoglobin subunit delta, hemoglobin subunit alpha, hemoglobin subunit beta, Glutathione S-transferase P, Cystatin-A, PEBP1, HSPA2, TUBB3, DNAL1, and Krt14;
(ii) comparing the expression level of the protein biomarker in the subject to a reference or control expression level for the at least one or more or each biomarker; and
(iii) diagnosing PD in the subject on the basis of the comparison, wherein the change in the expression level of protein biomarker or the ratio thereof of the subject's expression level from those of reference or control expression level correlates with a diagnostic evaluation of PD.

13. The method according to claim 12, wherein measuring the expression level of the one or more protein biomarker is measured in a sample obtained from the subject, and wherein the sample is an olfactory cleft mucus sample.

14. The method according to claim 12, wherein measuring and comparing the expression level of the one or more protein biomarker is measuring and comparing the expression level of alpha-synuclein (α-Syn), DJ-1, and/or MCP1.

15. The method according to claim 14, wherein comparing the expression level of the one or more protein biomarker is comparing the expression level ratio of α-Syn/DJ-1.

16. The method according to claim 12, wherein a ratio of α-Syn/DJ-1 of greater than 1.5 correlates with a diagnosis of PD in the subject.

17. The method according to claim 21, wherein diagnosing PD in the subject comprises early-stage PD diagnosis.

18. The method according to claim 12, wherein measuring the expression level of the one or more protein biomarker is measuring using a Western Blot (WB), a Luminex-based multiplex immunoassay, an enzyme linked immunoabsorbent assay (ELISA), an immunoprecipitation assay, a complement fixation assay, a fluorescence activated cell sorter (FACS), a protein chip, or a Liquid Chromatography with tandem mass spectrometry (LC-MS/MS).

19. The method according to claim 12, wherein the change in the expression level of protein biomarker or the ratio thereof comprises an upregulation of the one or more of protein biomarker in comparison to said reference or control or a downregulation of one or more selected genes in comparison to said reference or control.

20. (canceled)

21. The method of claim 12 treating the subject with PD modifying therapies.

22-23. (canceled)

24. The method according to claim 21, wherein the PD modifying therapies prevent and/or alleviate PD related symptoms including essential tremor, multiple system atrophy and progressive supranuclear palsy.
